(19) United States
(12) Patent Application Publication
(10) Pub. No.: US 2023/0346866 A1
(43) Pub. Date: Nov. 2, 2023

(54) COMPOSITIONS AND METHODS FOR TREATING AND PREVENTING NON-MALIGNANT RESPIRATORY DISEASE

(71) Applicant: NOVEL CONCEPTS MEDICAL LTD, Tel Aviv (IL)

(72) Inventor: Rachel ALKALAY, Tel Aviv (IL)

(73) Assignee: NOVEL CONCEPTS MEDICAL LTD, Tel Aviv (IL)

(21) Appl. No.: 17/912,633

(22) PCT Filed: Mar. 19, 2021
(86) PCT No.: PCT/IL2021/050308

§ 371 Date: Sep. 19, 2022

Related U.S. Application Data
(60) Provisional application No. 62/992,276, filed on Mar. 20, 2020.

(51) Int. Cl. A61K 36/53; A61K 36/258; A61K 36/324; A61K 36/424; A61K 36/71; A61K 36/738; A61K 36/85; A61K 38/48; A61P 31/14

(52) U.S. Cl. CPC A61K 36/53; A61K 36/258; A61K 36/324; A61K 36/424; A61K 36/71; A61K 36/738; A61K 36/85; A61K 38/4873; A61P 31/14

(57) ABSTRACT

A method of preventing or treating non-malignant respiratory disease (NMRD) in a subject in need thereof is provided. The method comprising administering to the subject an effective amount of a plant species or genus thereof-derived component selected from the group consisting of a plant part, extract thereof, fraction thereof, active ingredient thereof, synthetic analog thereof, mimetic thereof or combination thereof, wherein the component is capable of ameliorating symptoms of NMRD and wherein the plant species is selected from the group consisting of Nigella sativa, Thymus capitatus, Thymus vulgaris, Origanum syriacum, Thymbra spicata, Satujera thymbra, Sesamum indicum, Rhus coriaria, Gynostemma polyphyllum, Boswellia sacra and Panax ginseng preventing or treating NMRD in the subject.

### FIELD AND BACKGROUND OF THE INVENTION

The present invention, in some embodiments thereof, relates to compositions and methods for treating and preventing non-malignant respiratory disease (NMRD).

Non-malignant respiratory disease (“NMRD”, also known as non-malignant pulmonary disease or “NMPD”) is a diverse group of acute and chronic conditions affecting the respiratory tract and interfering with gas exchange which constitute a common and significant cause of illness and death worldwide, and make up the vast majority of ICU utilization throughout all hospital systems. Chronic lung disease of some form, including, inter alia, Interstitial Lung Disease (ILD), Bronchiectastis and Chronic Obstructive Pulmonary Disease (COPD), affects greater than 10% of individuals in both highly industrialized and less highly industrialized nations, with chronic obstructive pulmonary disease being the most common.

Chronic respiratory disease is of particular importance in Environmental and Occupational Medicine, with environmental (e.g. pollution) and workplace exposures contributing to the burden of disease across a range of nonmalignant lung conditions in the young, in adults and in the elderly (in addition to the 100% burden for the classic occupational pneumoconioses). This burden has important clinical, research, and policy implications. Current treatment for chronic respiratory disease focuses mostly on palliative care.

According to the World Health Organization (WHO) study, in low-income countries pneumonia leads the top five death causing diseases followed by heart disease, diarrhea, HIV/AIDS and stroke, in high-income countries, pneumonia, and asthma/bronchitis are closing the top five death causing diseases topped only by heart disease, stroke and lung cancer. (see who.int/news/item/27-10-2008-new-study-presents-state-of-the-world-s-health).

Thus, there is great need for a new therapeutic and preventive treatment for non-malignant respiratory disease.

### SUMMARY OF THE INVENTION

According to an aspect of the invention there is provided a method of preventing or treating non-malignant respiratory disease (“NMRD”) in a subject in need thereof, the method comprising administering to the subject an effective amount of a plant species or genus thereof-derived component selected from the group consisting of a plant part, extract thereof, fraction thereof, active ingredient thereof, synthetic analog thereof, mimetic thereof or combination thereof, wherein the component is capable of ameliorating symptoms of NMRD and wherein the plant species is selected from the group consisting of Nigella sativa, Thymus capitatus, Thymus vulgaris, Origanum syriacum, Thymbra spicata, Satujera thymbra, Sesamum indicum Rhus coriaria, Gynostemma pentaphyllum, Boswellia sacra and Panax ginseng, preventing or treating NMRD in the subject.

According to an aspect of the invention there is provided a vaccine against a NMRD comprising an effective amount of a plant species or genus thereof-derived component selected from the group consisting of a plant part, extract thereof, fraction thereof, active ingredient thereof, synthetic analog thereof, mimetic thereof or combination thereof, wherein the component is capable of ameliorating symptoms of NMRD and wherein the plant species is selected from the group consisting of Nigella sativa, Thymus capitatus, Thymus vulgaris, Origanum syriacum, Thymbra spicata, Satujera thymbra, Sesamum indicum Rhus coriaria, Gynostemma pentaphyllum, Boswellia sacra and Panax ginseng.

According to an aspect of the invention there is provided a pharmaceutical composition comprising an effective amount of a plant species or genus thereof-derived component selected from the group consisting of a plant part, extract thereof, fraction thereof, active ingredient thereof, synthetic analog thereof, mimetic thereof or combination thereof, wherein the component is capable of ameliorating symptoms of NMRD and wherein the plant species is selected from the group consisting of Nigella sativa, Thymus capitatus, Thymus vulgaris, Origanum syriacum, Thymbra spicata, Satujera thymbra, Sesamum indicum, Rhus coriaria, Gynostemma pentaphyllum, Boswellia sacra and Panax ginseng for use in preventing or treating NMRD.

According to an aspect of the invention there is provided a composition of matter comprising at least 2 of a plant species or genus thereof-derived components selected from the group consisting of a plant part, extract thereof, fraction thereof, active ingredient thereof, synthetic analog thereof, mimetic thereof or combination thereof, wherein the component is capable of ameliorating symptoms of NMRD and wherein the plant species is selected from the group consisting of Nigella sativa, Thymus capitatus, Thymus vulgaris, Origanum syriacum, Thymbra spicata, Satujera thymbra, Sesamum indicum, Rhus coriaria, Gynostemma pentaphyllum, Boswellia sacra and Panax ginseng.

According to an aspect of the invention there is provided a food supplement comprising a combination of at least 2 of a plant species or genus thereof-derived component selected from the group consisting of a plant part, extract thereof, fraction thereof, active ingredient thereof, synthetic analog thereof, mimetic thereof or combination thereof, wherein the component is capable of ameliorating symptoms of NMRD and wherein the plant species is selected from the group consisting of Nigella sativa, Thymus capitatus, Thymus vulgaris, Origanum syriacum, Thymbra spicata, Satujera thymbra, Sesamum indicum, Rhus coriaria, Gynostemma pentaphyllum, Boswellia sacra and Panax ginseng.

According to an aspect of the present invention there is provided compositions or food supplements comprising Bromelain or pineapple extracts comprising Bromelain.

According to some embodiments of the invention the NMRD is selected from the group pulmonary hypertension, interstitial lung disease, acute lung injury (ALI), acute respiratory distress syndrome (ARDS), sarcoidosis of the lung, bronchiectasis, asbestosis, berylliosis, silicosis, anthracosis, Histiocytosis X, pneumotitis, smoker's lung, bronchiolitis obliterans, lung scarring due to tuberculosis and pulmonary fibrosis, chronic obstructive pulmonary disease, pneumoconiosis, traumatic pulmonary injury, pulmonary infections and pulmonary manifestations of connective tissue diseases, including systemic lupus erythematosus, rheumatoid arthritis, scleroderma, polymyositis and dermatomyositis.

According to some embodiments of the invention the NMRD is Chronic Obstructive Pulmonary Disease (COPD), Idiopathic Pulmonary Fibrosis (IPF) or Asthma.

According to some embodiments of the invention the symptoms are selected from the group consisting of coughing, shortness of breath, wheezing, difficulty in breathing, inflammation of the airways, chest tightness and exercise intolerance.

According to some embodiments of the invention, the component comprises at least 2 components.

According to some embodiments of the invention, the component comprises at least 3 components.

According to some embodiments of the invention, the component comprises at least 4 components.

According to some embodiments of the invention, the component comprises at least 5 components.

According to some embodiments of the invention, the component comprises 5-10 components.

According to some embodiments of the invention, the component comprises thymoquinone or an analog thereof.

According to some embodiments of the invention, the component comprises thymol or an analog thereof.

According to some embodiments of the invention, the component comprises carvacrol or an analog thereof.

According to some embodiments of the invention, the component comprises Tryptophan or an analog thereof.
According to an aspect of the invention there is provided a food supplement, composition or extracts further including “Beduin Tea” comprising
Rose Leaves Micromeria fruticose, Salvia, cymbopgon (Citral) Aloysia, Verbena officinalis, Origanum majorana, menthe
According to an aspect of the invention there is provided a food supplement, composition or extracts further including “Beduin Tea” comprising
Thyme, sage, cardamom, cinnamon, black tea, habuk, Marmaya.
Further details of components of Thyme Vulgaris are included in APPENDIX1.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWING(S)

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIGS. 1A-C shows embodiments in plant extraction methods as taken from berkem(dot)com. FIG. 1A—scheme describing the general principle of plant extraction; FIG. 1B—scheme describing the main separation process according to some embodiments; FIG. 1C—scheme describing parameters that may influence the process.

FIG. 2—depicting SDS-page of the SARS-CoV-2 S1 subunit protein digestion assay with the tested extracts following an incubation time of 6 h at 37° c.

FIG. 3—depicting SDS-page of the SARS-CoV-2 S2 subunit protein digestion assay with the tested extracts following an incubation time of 6 h at 37° c.

FIG. 4—depicting SDS-page of the SARS-CoV-2 Nucleocapsid digestion assay with the tested extracts following an incubation time of 6 h at 37° c.

FIG. 5—depicting a graphic representation of the densitometry test of the SARS-CoV-2 S1 subunit protein digestion assay with the tested extracts following an incubation time of 6 h at 37° c.

FIG. 6—depicting a graphic representation of the densitometry test of the SARS-CoV-2 S2 subunit protein digestion assay with the tested extracts following an incubation time of 6 h at 37° c.

FIG. 7—depicting a graphic representation of the densitometry test of the SARS-CoV-2 Nucleocapsid digestion assay with the tested extracts following an incubation time of 6 h at 37° c.

### DESCRIPTION OF SPECIFIC EMBODIMENTS OF THE INVENTION

The present invention, in some embodiments thereof, relates to compositions and methods for treating and preventing non-malignant respiratory disease (NMRD).

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

Non-malignant respiratory disease (NMRD) is a group of acute and chronic conditions affecting the respiratory tract and interfering with gas exchange, exclusive of cancerous and malignant proliferative pulmonary disease. In mild forms (e.g. the common cold, mild bronchitis, pharyngitis, mild allergic respiratory distress), symptoms of NMRD may include coughing, wheezing, pulmonary and/or nasal congestion, copious mucus and mild shortness of breath. Most often, patients with mild NMRD will experience mild to moderate symptoms and recover without requiring special treatment. Moderate to severe cases of NMRD, and particularly in older individuals, those with underlying medical problems and individuals frequently exposed to common triggers of respiratory disease such as particulate pollution (e.g. coal dust, asbestos, silica, etc), dust mites and allergens are more likely to develop into serious, chronic illness, in many cases requiring intensive care with ventilation and life-threatening disease. At this time, there are no specific vaccines or effective preventative treatments for many of the non-malignant respiratory conditions.

Thus, according to an aspect of the invention there is provided a method of preventing or treating a non-malignant respiratory disease or condition (NMRD) in a subject in need thereof, the method comprising administering to the subject an effective amount of a plant species or genus thereof-derived component selected from the group consisting of a plant part, extract thereof, fraction thereof, active ingredient thereof, synthetic analog thereof, mimetic thereof or combination thereof, wherein the component is capable of ameliorating symptoms of NMRD and wherein the plant species is selected from the group consisting of Nigella sativa, Thymus capitatus, Thymus vulgaris, Origanum syriacum, Thymbra spicata, Satujera thymbra, Sesamum indicum, Rhus coriaria, Gynostemma pentaphyllum, Boswellia sacra and Panax ginseng, preventing or treating NMRD in the subject.

According to an alternative or an additional aspect of the invention there is provided a vaccine against a NMRD comprising an effective amount of a plant species or genus thereof-derived component selected from the group consisting of a plant part, extract thereof, fraction thereof, active ingredient thereof, synthetic analog thereof, mimetic thereof or combination thereof, wherein the component is capable of ameliorating symptoms of NMRD and wherein the plant species is selected from the group consisting of Nigella sativa, Thymus capitatus, Thymus vulgaris, Origanum syriacum, Thymbra spicata, Satujera thymbra, Sesamum indicum, Rhus coriaria, Gynostemma pentaphyllum, Boswellia sacra and Panax ginseng.

Plant derived component, synthetic analogs and the like are well established compositions used to treat a wide spectrum of diseases and syndromes. From the Aspirin (acetyl salicylic acid) historically discovered as a component of Salix alba to the Vinca derived chemotherapeutic drugs vinblastine and vincristine. It stands to reason that plant derived components and substances are capable of attenuating a wide spectrum of viral infections and ameliorating the same. Moreover, the aforementioned plant substances maybe used as a profilaxis for human but for livestock as well, thus lowering the rate of zoonosis. Some representative viruses, viral families and viral genus are depict in table A.

TABLE A

| FAMILY | GENUS | DISEASE/VIRUS | ABBREVIATION |
|---|---|---|---|
| Adenoviridae | | Respiratory | HAdV-B/C |
| | | Adenovirus | |
| Arenaviridae | Mammarenavirus | Lassa Virus | LASV |
| Astroviridae | Mamstrovirus | Mamstrovirus | MAstV |
| Caliciviridae | | Norovirus | |
| Coronaviridae | | Severe Acute | SARS-CoV |
| | | Respiratory | |
| | | Syndrome | |
| | | Middle East | MERS-CoV |
| | | Respiratory | |
| | | Syndrome | |
| | | Covid-19 | SARS-CoV-2 |
| Filoviridae | Ebolavirus | Ebola Virus | EVD |
| | Marburgvirus | Marburg Virus | marv |
| Flaviviridae | Flavivirus | Dengue Fever | DENV |
| | Flavivirus | Japanese Encephalitis | JEV |
| | Flavivirus | Powassan Virus | POWV |
| | Flavivirus | West Nile Virus | WNV |
| | Flavivirus | Yellow Fever | |
| | Flavivirus | Zika Virus | ZIKV |
| Hantaviridae | Orthohantavirus | Hanta Virus | HPS |
| | | Pulmonary Syndrome | |
| Herpesviridae | | Herpes Simplex | HSV |
| | | Varicella Zoster | VZV |
| | | Cytomegalovirus | CMV |
| | | Epstein-Barr Virus | EBV |
| | | Roseola Virus | HHV-6 |
| Matonaviridae | Rubivirus | Rubella (German | RUBV |
| | | Measles) | |
| Nairoviridae | Orthornavirus | Crimean-Congo | CCHFV |
| | | Hemorrhagic Fever | |
| Papillomaviridae | | Human Papilloma | HPV |
| | | Virus | |
| Paramyxoviridae | Morobilivirus | Measles | MeV |
| | Orthobulavirus | Mumps | MuV |
| | Henipavirus | Nipah Virus | NiV |
| Peribunyviridae | Orthobunyavirus | Cache Valley | CVV |
| | | Orthobunyavirus | |
| Picornaviridae | | Poliomyelitis | Polio |
| | | Coxsackie Virus | CV |
| | | Echovirus | EV |
| | | Hepatitis A, B, C, D, E | HAV, HBV, HCV, HDV, HEV |
| Pneomoviridae | Orthopneomovirus | Respiratory Syncytial Virus | RSV |
| | Metapneumovirus | Human | HMPV |
| | | Metapneumovirus | |
| Poxviridae | Orthopoxviros | Variola (Small Pox) | VARV |
| Reoaviridae | Rotavirus | Rota Virus | |
| Rhabdoviridae | Lyssavirus | Rabies | RARV |
| Retroviridae | Lentivirus | Human | HIV 1/HIV 2/AIDS |
| | | immunodeficiency | |
| | | virus | |
| Togaviridae | Alphavirus | Chinkungunya | CHIKV |

According to an alternative or an additional aspect of the invention there is provided a pharmaceutical composition comprising an effective amount of a plant species or genus thereof-derived component selected from the group consisting of a plant part, extract thereof, fraction thereof, active ingredient thereof, synthetic analog thereof, mimetic thereof or combination thereof, wherein the component is capable of ameliorating symptoms of NMRD and wherein the plant species is selected from the group consisting of Nigella sativa, Thymus capitatus, Thymus vulgaris, Origanum syriacum, Thymbra spicata, Satujera thymbra, Sesamum indicum, Rhus coriaria, Gynostemma pentaphyllum, Boswellia sacra and Panax ginseng for use in preventing or treating a NMRD.

According to an alternative or an additional aspect of the invention there is provided a composition of matter comprising at least 2 of a plant species or genus thereof-derived components selected from the group consisting of a plant part, extract thereof, fraction thereof, active ingredient thereof, synthetic analog thereof, mimetic thereof or combination thereof, wherein the component is capable of ameliorating symptoms of NMRD and wherein the plant species is selected from the group consisting of Nigella sativa, Thymus capitatus, Thymus vulgaris, Origanum syriacum, Thymbra spicata, Satujera thymbra, Sesamum indicum, Rhus coriaria, Gynostemma pentaphyllum, Boswellia sacra and Panax ginseng.

The term “plant” as used herein encompasses whole plants, a grafted plant, ancestors and progeny of the plants and plant parts, including seeds, flowers, bark, shoots, stems, roots (including tubers), fruit, rootstock, scion, and plant cells, tissues and organs.

According to a specific embodiment, the plant part is a seed.

According to a specific embodiment, the plant part is a fruit.

According to a specific embodiment, the plant part is a leaf.

According to a specific embodiment, the plant part is a stem.

According to a specific embodiment, the plant part is a flower.

The plant part can be a solid part or a non-solid part such as oil or aqueous portions of the plant.

The plant may be in any form including suspension cultures, embryos, meristematic regions, callus tissue, leaves, gametophytes, sporophytes, pollen, and microspores.

The term plant refers to a wild plant or a cultivated variety thereof.

As used herein the term “plant species” refers to a sub-group of one or more plants within the genus. These plants will share similar characteristics with each other. There may be a single plant within a species, or there may be many hundreds of plants. The term intends to include subspecies, such as grown or can be found in different geographical location, e.g., Lebanese Sumac and Syrian Sumac or Korean ginseng and American ginseng.

As used herein “plant genus” refers to a taxonomic rank below family and above species.

It will be appreciated that the relevant species and genera and listed below and each option or combination thereof represents a different embodiment of the invention.

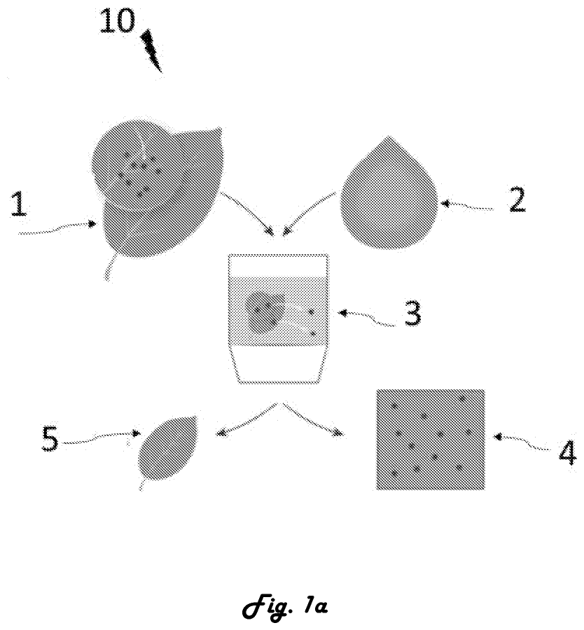

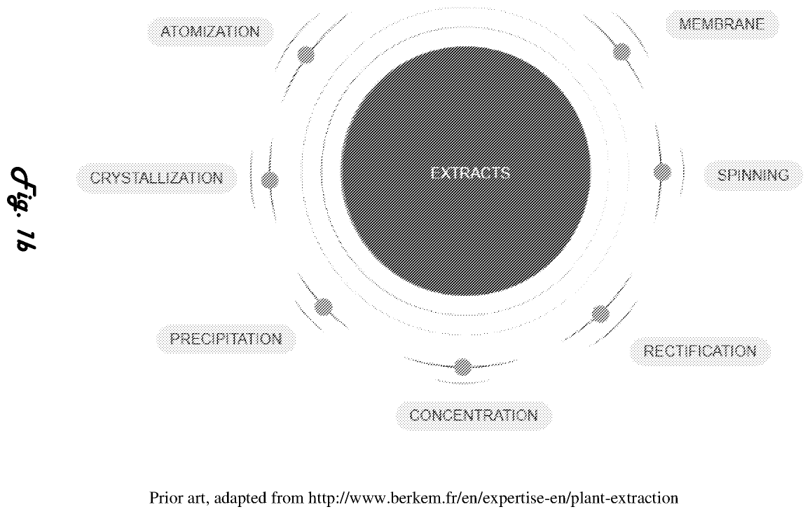

The term “extraction” refers to a separation process which relies on the separation of one or more analytes from the components of a sample other than the one or more analytes. Extractions are processes that typically use two immiscible phases to separate one or more solutes from one phase into the other. The distribution of a solute between two phases is an equilibrium condition described by partition theory. For example, boiling tea leaves in water extracts the tannins, theobromine, and caffeine out of the leaves and into the water. More typical extractions preformed typically but not only in a laboratory are settings of organic compounds out of an aqueous phase and into an organic phase. Common extractants are arranged from ethyl acetate to water (ethyl acetate<acetone<ethanol<methanol<acetone:water (7:3)<ethanol:water (8:2)<methanol:water (8:2)<water) in increasing order of polarity according to the Hildebrand solubility parameter. Procedures for plant extraction are provided in FIGS. 1A-C.

The term “extract” as used herein refers to the result of such process of separation that can take the form of a solution formulation or other chemical form depending on the extraction process. In particular, the term extract can relate to a substance made by extracting a part of a sample (e.g. a raw material), such as by using a solvent such as ethanol or water. In various instances an extract relates to a solvent that is enriched in one or more solute. In particular, a “plant extract” in the sense of the present disclosure typically comprises a concentrated preparation of a plant material obtained by isolating or purifying desired active constituents with one or more extraction processes.

The choice of the solvent depends on the desired component to be obtained. For example, to extract polar components in an extraction process suggested solvents include, but are not limited to, water, ethanol methanol or butanol while for non polar compounds diethyl ether, hexane or chloroform depending on the use of the extract. For midpolar one may choose Ethyl acetate but other solvents can be used as well.

The general procedure of solid/liquid extraction can be scaled in five different ways:

Maceration: the contact stage is maintained at room temperature.

Decoction or reflux: the contact stage is maintained at the boiling point of the solvent.

Digestion: the contact stage is maintained at a temperature in between those of the previous two cases.

Infusion: the boiling solvent is poured over the solid, then left to cool for a set time.

Leaching or percolation: the solvent passes through the biomass.

It is also possible to combine these methods with each other or with other processes such as distillation, steam distillation, rectification, etc.

According to another embodiment, the use of various solvents, either successively or in combination is contemplated and the ordinary skilled of organic chemistry will know which to choose according to the active ingredient as described below.

Extraction may be further assisted by other means such as ultrafiltration, reverse osmosis, high pressure (supercritical CO2), microwaves, ultrasound, etc.

In some embodiments, the plant part is contacted with a polar solvent (e.g. ethanol) or nonpolar solvent (e.g., hexane or pentane) for several minutes, e.g., 15 minutes or more, about 30 minutes or more, about 1 hour or more, about 2 hours or more, or about 5 hours or more.

Temperature can also be controlled during the contacting.

According to specific embodiments, the plant part is contacted with the solvent (e.g. ethanol) while being constantly mixed e.g. on a shaker.

It will be appreciated that the extraction process can also be solvent-free.

For example, solvent-free microwave extraction (SFME) has been proposed as a green method for the extraction of essential oil from aromatic herbs that are extensively used in the food industry. This technique is a combination of microwave heating and dry distillation performed at atmospheric pressure without any added solvent or water. The isolation and concentration of volatile compounds is performed in a single stage. In some embodiments, SFME and/or hydro-distillation (HD)), are used for the extraction of essential oil from the plants of the invention.

In some embodiments, the process of the present invention comprises isolating a liquid extract (i.e. filtered extract) from the mixture (i.e. crude extract) comprising the liquid extract and solids. Suitable means for isolating the liquid extract (i.e. filtered extract) include those known in the art of organic synthesis and include, but are not limited to, gravity filtration, suction and/or vacuum filtration, centrifuging, setting and decanting, and the like. In some embodiments, the isolating comprises filtering a liquid extract through a porous membrane, syringe, sponge, zeolite, paper, or the like having a pore size of about 1-5 μm, about 0.5-5 μm, about 0.1-5 μm, about 1-2 μm, about 0.5-2 μm, about 0.1-2 μm, about 0.5-1 μm, about 0.1-1 μm, about 0.25-0.45 μm, or about 0.1-0.5 μm (e.g. about 2 μm, about 1 μm, about 0.45 μm, or about 0.25 μm).

According specific embodiments, the present invention contemplates drying (i.e. removal of the polar/non-polar solvent) and/or freezing the filtered extract following generation thereof.

The method for drying the filtered extract (i.e. removing the polar solvent) is not particularly limited, and can include solvent evaporation at a reduced pressure (e.g., sub-atmospheric pressure) and/or an elevated temperature (e.g., above about 25° C.). In some embodiments, it can be difficult to completely remove a solvent from a liquid extract by standard solvent removal procedures such as evaporation. In some embodiments, processes such as co-evaporation, lyophilization, and the like can be used to completely remove the polar solvent from a liquid fraction to form a dry powder, dry pellet, dry granulate, paste, and the like. According to a specific embodiment the polar solvent is evaporated with a vacuum evaporator.

The selection of the extraction process much depends on the component to be isolated.

It will be appreciated that following generation of the extract, specific embodiments of the present invention further contemplate additional purification steps so as to further isolate/purify active agents from the extract, for example, by fractionating the filtered extract.

As used herein “a fraction” refers to a portion of the extract that contains only certain chemical ingredients of the extract but not all.

Fractionating can be performed by processes such as, but not limited to: column chromatography, preparative high performance liquid chromatography (“HPLC”), reduced pressure distillation, and combinations thereof.

According to a specific embodiment, fractionating is performed by HPLC.

In some embodiments, fractionating comprises re-suspending the filtered extract in a polar solvent (such as methanol, as discussed above), applying the polar extract to a separation column, and isolating the extract having the anti-respiratory disease (e.g. anti-fibrotic, anti-inflammatory) activity by column chromatography (preparative HPLC).

An eluting solvent is applied to the separation column with the polar extract to elute fractions from the polar extract. Suitable eluting solvents for use include, but are not limited to, methanol, ethanol, propanol, acetone, acetic acid, carbon dioxide, methylethyl ketone, acetonitrile, butyronitrile, carbon dioxide, ethyl acetate, tetrahydrofuran, di-iso-propylether, ammonia, triethylamine, N,N-dimethylformamide, N,N-dimethylacetamide, and the like, and combinations thereof.

According to an alternative or an additional embodiment, liquid chromatography comprises high performance liquid chromatography (HPLC).

According to an alternative or an additional embodiment, liquid chromatography is performed on a reverse stationary phase.

The fractions may be characterized by analytical methods such as, but not limited to, spectroscopic methods such as, but not limited to, ultraviolet-visible spectroscopy (“UV-Vis”), infrared spectroscopy (“IR”), and the like; mass-spectrometry (“MS”) methods such as, but not limited to, time-of-flight MS; quadrupole MS; electrospray MS, Fourier-transform MS, Matrix-Assisted Laser Desorption/Ionization (“MALDI”), and the like; chromatographic methods such as, but not limited to, gas-chromatography (“GC”), liquid chromatograph (“LC”), high-performance liquid chromatography (“HPLC”), and the like; and combinations thereof (e.g., GC/MS, LC/MS, HPLC/UV-Vis, and the like), and other analytical methods known to persons of ordinary skill in the art.

The component (active ingredients, extract and/or fractions) obtained may be tested for reducing NMRD or symptoms thereof. Exemplary methods for testing the effect are further described herein below as well as in the Examples section which follows.

The active ingredients, extract and/or fraction described herein may be immediately used or stored until further used.

According to specific embodiments, the active ingredients, extract and/or fractions is kept frozen, e.g. in a freezer, until further use (e.g. at about −20° C. to −90° C., at about −70° C. to −90° C., e.g. at −80° C.), for any required length of time.

According to other specific embodiments, the active ingredients, extract and/or fractions is immediately used (e.g. within a few minutes e.g., up to 30 minutes).

The active ingredients, extract and/or fractions may be used separately. Alternatively, different active ingredients, extract and/or fractions (e.g. from different plants or from separate extraction procedures) may be pooled together. Likewise, different active ingredients, extract and/or fractions (from the same extract, from different extracts, from different plants and/or from separate extraction procedures) may be pooled together.

Using the present teachings, the present inventor was able to identify not only plants and extracts that can be used to effectively treat or prevent NMRD, but also active ingredients thereof.

“Active ingredient” refers to a defined chemical composition which is responsible for the anti (preventive or therapeutic) effect against NMRD.

The active ingredient can be purified from a plant or chemically synthesized (artificial, man-made).

Also contemplated herein are analogs and derivatives of the active ingredients as long as the anti (preventive or therapeutic) effect against NMRD is maintained (see e.g., Examples section which follows), which are also referred to as mimetics.

Following are some non-limiting examples for extraction of active ingredients from selected plants of the present invention.

Extraction from leaves of T. capitatus—The Aerial parts of T. capitatus (leaves) samples are collected. Leaves separated from branches are dehydrated at room temperature for 7 days and slightly blended into fine powders for extractions.

Essential oil (EO) extraction—hydro-distillation is used to extract EO from the plant, e.g., dried aerial parts of T. capitatus. In brief, the extraction is conducted for several hours for example, 3 h, by mixing 100 g of plants in 500 mL of distilled water. The extract is dried and concentrated using sodium sulphate and rotatory evaporator under reduced pressure. The EO yield is established by quantity of the obtained oil in mL for 100 g of dried plant. Finally, the pure EO os stored at −4° C. until further analyzed.

Essential oil analysis—The chemical composition of EO is examined by GC and GC-MS. GC analysis is conducted using gas chromatograph. The proportion of the constituents is determined by the integration of peak areas. In addition, mass spectrometry (MS) can be used to analyze the EO typically under the same conditions as described above for gas chromatography. The identification of the different compounds is defined by comparison of their retention indexes (determined relatively to the retention times of a series of n-alkanes) with those of standards of the Wiley library search routines12, based on fit and purity of mass spectra. Such conditions are used for determining the active ingredients as described below.

Extraction from Satujera thymbra:

Air dried aerial parts from S. thymbra were collected in Lebanon at random during April 2009. For 3 h the plant material was submitted to steam distillation using a clevenger-type apparatus to produce the essential oil with a yield of 0.84% (w/w). Oil is dried using anhydrous magnesium sulfate and stored at 4° C. S. thymbra oil was analyzed by GC/MS.

Extraction from Rhus coriaria (Sumac)

In order to isolate, determine and identify the compounds from the Rhus coriaria fruits, different extracts are taken from the fruit or leaves of the Sumac plant. Some are isolated from aqueous extracts, others from alcoholic extracts and some from lipid extracts. Hydrolysable tannins compose the highest percentage in the Sumac fruits, followed by flavonoids. This emphasizes the antioxidant potential of the fruit. Following hydrolysable tannins, comprising almost 20% of the fruit's mass, are other unidentified compounds. Subsequently there are anthocyanins, isoflavonoids, terpenoids and diterpenes. Analysis of the chemical properties of sumac fruit is conducted on ripe fruits and have found a 2.6% protein content, 7.4% fat content, 14.6% fiber content, 1.8% ash. Also, a calorimetric calculation showed that 100 g of sumac fruit contains 147.8 kcal.

Extraction of Thymoquinone from Nigella sativa

Various methods can be used including microwave-assisted extraction system having temperature controlling function as well as other extraction methods, Soxhlet and conventional solid/liquid extraction.

Nigella Sativa

| 24-ETHYL-LOPHENOL | Seed Oil | | |
|---|---|---|---|
| 24-METHYL-LOPHENOL | Seed Oil | | |
| 24-METHYLENE-CYCLOARTANOL | Seed Oil | | |
| 5-DEHYDRO-AVENASTEROL | Seed Oil | | |
| 7-DEHYDRO-AVENASTEROL | Seed Oil | | |
| ALANINE | Seed | 8,000 | 10,255 |
| ALPHA-SPINASTEROL | Seed Oil | | |
| | Seed | | |
| ARABIC-ACID | Seed | | |
| ARACHIDIC-ACID | Seed Oil | | 1,900 |
| ARACHIDONIC-ACID | Seed Oil | | 24,900 |
| ARGININE | Seed | 41,500 | 53,050 |
| ASCORBIC-ACID | Leaf | | 2,577 |
| ASH | Seed | 38,000 | 53,000 |
| ASPARAGINE | Seed | | 0 |
| ASPARTIC-ACID | Seed | 10,670 | 13,650 |
| ASTRAGALIN | Seed | | 200 |
| BETA-AMYRIN | Seed Oil | | |
| BETA-SITOSTEROL | Seed Oil | | |
| | Seed | | 3,218 |
| BUTYROSPERMOL | Seed Oil | | |
| CALCIUM | Seed | | 10,600 |
| CAMPESTANOL | Seed Oil | | |
| CAMPESTEROL | Seed Oil | | |
| | Seed | | |
| CARBOHYDRATES | Seed | | 339,600 |
| CARVONE | Seed | | |
| | Essent. Oil | | |
| | Seed | 2,250 | 9,600 |
| CHOLESTEROL | Seed Oil | | |
| | Seed | | |
| CITROSTADIENOL | Seed Oil | | |
| CYCLOARTENOL | Seed Oil | | |
| CYCLOEUCALENOL | Seed Oil | | |
| CYMENE | Seed | | |
| CYSTINE | Seed | | |
| D-LIMONENE | Seed | | |
| DAMASCENINE | Plant | | |
| DEHYDROASCORBIC-ACID | Leaf | | 295 |
| DITHYMOQUINONE | Seed Oil | | |
| EICOSADIENOIC-ACID | Seed Oil | | 25,000 |
| | Seed | 8,979 | 10,525 |
| EO | Seed Oil | | 4,500 |
| | Seed | 4,000 | 16,000 |
| FAT | Seed | 354,900 | 416,000 |
| FIBER | Seed | | 55,000 |
| FIXED-OIL | Seed | | 380,000 |
| GLUCOSE | Seed | | |
| GLUTAMIC-ACID | Seed | 28,085 | 35,900 |
| GLYCINE | Seed | 8,840 | 20,700 |
| GRAMISTEROL | Seed Oil | | |
| HEDERAGENIN | Seed | | 10,000 |
| INDOLE-3-ACETIC-ACID | Tissue | | 14 |
| | Culture | | |
| IRON | Seed | | 140 |
| ISOLEUCINE | Seed | 8,570 | 10,960 |
| KAEMPFEROL-3-O-BETA-GLUCOPYRANOSYL-(1,2)-O-BETA- | Seed | | 100 |
| GALACTOPYRANOSYL-(1,2)-BETA-GLUCOPYRANOSIDE | | | |
| LEUCINE | Seed | 23,130 | 29,595 |
| LINOLEIC-ACID | Seed Oil | 487,600 | 576,300 |
| | Seed | 128,124 | 233,459 |
| LINOLENIC-ACID | Seed Oil | 5,700 | 7,000 |
| | Seed | 2,484 | 2,912 |
| LIPASE | Seed | | |
| LOPHENOL | Seed Oil | | |
| LYSINE | Seed | 16,200 | 20,700 |
| MELANTHIGENIN | Seed | | |
| MELANTHIN | Plant | | 15,000 |
| | Seed | | 15,000 |
| METHIONINE | Seed | 13,100 | 16,750 |
| MYRISTIC-ACID | Seed Oil | 1,900 | 18,000 |
| | Seed | 567 | 1,082 |
| NIGELLICINE | Seed | | |
| NIGELLIDINE | Seed | | |
| NIGELLIMINE | Seed | | 0.13 |
| NIGELLIMINE-N-OXIDE | Seed | | 0.2 |
| NIGELLIN | Seed | | |
| NIGELLINE | Seed | | |
| | Essent. Oil | | |
| NIGELLONE | Seed | | |
| | Essent. Oil | | |
| | Plant | | |
| | Seed | | |
| OBTUSIFOLIOL | Seed Oil | | |
| OLEIC-ACID | Seed Oil | 244,600 | 262,400 |
| | Seed | 89,911 | 184,912 |
| PALMITIC-ACID | Seed Oil | 120,000 | 171,200 |
| | Seed | 22,464 | 50,523 |
| PALMITOLEIC-ACID | Seed Oil | | 2,000 |
| PHENYLALANINE | Seed | 16,850 | 21,560 |
| PHYTOSTEROLS | Seed | | 5,100 |
| POTASSIUM | Seed | | 5,820 |
| PROLINE | Seed | 11,350 | 14,520 |
| PROTEIN | Seed | 210,000 | 271,900 |
| QUERCETIN-3′-GLUCOSIDE | Seed | | 440 |
| QUERCETIN-3-O-(6-FERULOYL-BETA-GLUCOPYRANOSYL)-(1,2)- | Seed | | 240 |
| BETA-GALACTOPYRANOSYL-(1,2)-BETA-GLUCOPYRANOSIDE | | | |
| QUERCETIN-3-O-BETA-GLUCOPYRANOSYL-(1,2)-O-BETA- | Seed | | 1,380 |
| GALACTOPYRANOSYL-(1,2)-BETA-GLUCOPYRANOSIDE | | | |
| RESIN | Seed | | |
| RUTIN | Seed | | 200 |
| SERINE | Seed | 4,210 | 5,385 |
| SODIUM | Seed | | 980 |
| STEARIC-ACID | Seed Oil | 18,100 | 60,400 |
| | Seed | 8,722 | 10,192 |
| STIGMAST-7-EN-3-BETA-OL | Seed Oil | | |
| STIGMASTANOL | Seed Oil | | |
| STIGMASTEROL | Seed Oil | | |
| | Seed | | |
| TANNIN | Seed | | |
| TARAXEROL | Seed Oil | | |
| TELFAIRIC-ACID | Seed Oil | | |
| THREONINE | Seed | 2,615 | 3,345 |
| THYMOHYDROQUINONE | Seed | | |
| THYMOL | Seed Oil | | |
| THYMOQUINONE | Seed | | |
| | Essent. Oil | | |
| | Seed Oil | | |
| | Seed | | |
| TIRUCALLOL | Seed Oil | | |
| TRYPTOPHAN | Seed | | |
| TYROSINE | Seed | 12,925 | 16,530 |
| VALINE | Seed | 6,500 | 8,325 |

According to a specific embodiment, active ingredients (e.g., which can be obtained by supercritical carbon dioxide extraction method) include but are not limited to:

| Compound | RIₑₓₚ | RIₗᵢₜ | SFE 1 | SFE 2 | HD SFE | Identification |
|---|---|---|---|---|---|---|
| n-Nonane^{a} | 905 | 900 | 0.12 | — | — | RI, MS |
| Tricyclene | 926 | 926 | tr | — | — | RI, MS |
| Camphene | 953 | 953 | — | — | 1.64 | RI, MS |
| β-Pinene | 958 | 959 | — | — | 0.40 | RI, MS |
| 2,4,(10)-Thujadiene | 967 | 960 | 4.74 | 0.19 | — | RI, MS |
| Sabinene | 978 | 977 | 1.05 | — | — | RI, MS |
| β-Myrcene | 990 | 991 | 0.31 | — | — | RI, MS |
| 1,8-Cineole | 1013 | 1010 | — | — | 0.98 | RI, MS |
| α-Terpinene | 1025 | 1026 | 2.34 | — | — | RI, MS |
| Limonene | 1034 | 1034 | 0.18 | 0.38 | 1.03 | RI, MS |
| γ-Terpinene | 1054 | 1056 | 27.46 | 13.20 | 12.87 | RI, MS |
| cis-Sabinene hydrate | 1063 | 1068 | — | 0.38 | Tr | RI, MS |
| allo-Ocimenol^{a} | 1079 | 1071 | — | 0.11 | — | RI, MS |
| Linalool | 1087 | 1080 | 0.25 | 0.19 | — | RI, MS |
| Terpinolene | 1091 | 1088 | — | — | Tr | RI, MS |
| trans-Sabinene hydrate | 1099 | 1097 | 0.37 | — | — | RI, MS |
| Terpinen-1-ol^{a} | 1124 | 1120 | — | — | 0.11 | RI, MS |
| 1,5,8-p-Menthatriene^{a} | 1130 | 1135 | 0.43 | 0.38 | — | RI, MS |
| Borneol | 1152 | 1152 | — | — | 1.02 | RI, MS |
| Pinocarvone | 1167 | 1165 | 2.96 | 3.00 | — | RI, MS |
| trans-Dihydrocarvone | 1208 | 1202 | — | 0.19 | — | RI, MS |
| Dihydrocarvone^{a} | 1215 | 1214 | 0.37 | 2.06 | — | RI, MS |
| Ocimenone (E)^{a} | 1249 | 1239 | 1.54 | 1.50 | — | RI, MS |
| Thymoquinone | 1250 | 1250 | 35.05 | 33.12 | 38.41 | RI, MS, NMR |
| Thymol | 1283 | 1288 | 7.43 | 5.30 | 16.95 | RI, MS, NMR |
| Carvacrol | 1299 | 1299 | 1.98 | 1.73 | 0.81 | RI, MS |
| 2-Undecanone | 1312 | 1315 | — | — | 13.72 | RI, MS |
| n-Octyl isobutyrate^{a} | 1323 | 1326 | — | — | 0.12 | RI, MS |
| α-Longipinene | 1330 | 1334 | 0.26 | — | — | RI, MS |
| Citronellyl acetate^{a} | 1339 | 1336 | — | — | 0.50 | RI, MS |
| Thymohydroquinone methyl ether^{a} | 1353 | 1351 | — | — | Tr | RI, MS |
| Cyclosativene | 1367 | 1366 | — | — | 1.43 | RI, MS |
| α-Longicyclene | 1381 | 1380 | 0.43 | 5.25 | — | RI, MS |
| α-Copaene | 1385 | 1383 | 1.54 | 2.00 | 0.41 | RI, MS |
| α-Longifolene | 1391 | 1387 | — | — | 0.51 | RI, MS |
| (Z)-Caryophyllene^{a} | 1395 | 1395 | 0.23 | — | — | RI, MS |
| β-Caryophyllene | 1420 | 1417 | 2.89 | 5.07 | 4.80 | RI, MS |
| Thymohydroquinone dimethylether^{a} | 1429 | 1425 | 0.43 | — | — | RI, MS |
| Aromadendrene^{a} | 1437 | 1438 | — | — | 1.04 | RI, MS |
| Thymohydroquinone | 1515 | 1509 | 1.17 | 1.12 | 2.31 | RI, MS, NMR |
| Davanone^{a} | 1587 | 1586 | 0.31 | — | — | RI, MS |
| 8-Heptadecene^{a} | 1683 | 1680 | 1.23 | 1.13 | 0.86 | RI, MS |
| Dihydrofarnesyl acetate^{a} | 1841 | 1840 | 2.28 | 4.69 | — | RI, MS |
| Pimaradiene^{a} | 1934 | 1935 | 1.23 | 2.25 | — | RI, MS |
| Palmitic acid | 1947 | 1946 | 0.18 | — | — | RI, MS |
| Pimara-8(14),15-diene | 1968 | 1966 | 0.92 | — | — | RI, MS |
| Octadecanoic acid | 2145 | 2157 | 0.26 | 12.31 | — | RI, MS |
| Total identified | | | 99.94 | 95.55 | 99.92 | |

Group compounds:

| | | | |
|---|---|---|---|
| Quinones | 44.08 | 39.54 | 57.67 |
| Monoterpene hydrocarbons | 36.51 | 14.15 | 15.94 |
| Oxygenated monoterpenes | 7.47 | 9.16 | 17.14 |
| Sesquiterpene hydrocarbons | 5.35 | 12.32 | 8.19 |
| Oxygenated sesquiterpenes | 2.59 | 4.69 | — |
| Diterpenes | 2.15 | 2.25 | — |
| Alkane | 0.12 | — | — |
| Alkenes | 1.23 | 1.13 | 0.86 |
| Fatty acids | 0.44 | 12.31 | — |
| Fatty acid esters | — | — | 0.12 |

Additional plants that are contemplated herein are of the genus Nigella.

Nigella is a genus of 18 species of annual plants in the family Ranunculaceae, native to Southern Europe, North Africa, South Asia, Southwest Asia and Middle East. Common names applied to members of this genus are Nigella, devil-in-a-bush or love-in-a-mist.
Nigella arvensis
Nigella carpatha
Nigella damascena
Nigella degenii
Nigella deserti
Nigella doerfleri
Nigella elata
Nigella fumariifola
Nigella hispanica
Nigella latisecta
Nigella nigellastrum
Nigella orientalis
Nigella oxypetala
Nigella papillosa
Nigella sativa
Nigella segetalis
Nigella stricta
Nigella unguicularis

According to a specific embodiment the active ingredient is thymoquinone.

Additional plants containing thymoquinone include, but are not limited to:
Monarda fistulos (of the genus Monarda);
Satureja montana (of the genus Satureja);

Additional families containing thymoquinone include, but are not limited to:
Asteraceae—examples include, but are not limited to the subfamilies:
   Barnadesioideae Bremer & Jansen
   Carduoideae Sweet
   Cichorioideae Chevallier
   Corymbioideae Panero & Funk
   Famatinanthoideae S. E. Freire, Ariza & Panero
   Gochnatioideae Panero & Funk
   Gymnarrhenoideae Panero & Funk
   Hecastocleidoideae Panero & Funk
   Mutisioideae Lindley
   Pertyoideae Panero & Funk
   Stifftioideae Panero
      Wunderlichioideae Panero & Funk
Cupressaceae
   Cunninghamioideae
   Taiwanioideae
   Athrotaxidoideae
   Sequoioideae
   Taxodioideae
   Callitroideae
   Cupressoideae
   Incertae sedis
Lamiacea
Ranunculacea
   Hydrastidoideae
   Glaucidioideae
   Coptoideae
   Thalictroideae
   Ranunculoideae

List of plants that contain Carvacrol include, but are not limited to:
Monarda didyma
Nigella sativa
Origanum compactum
Origanum dictamnus
Origanum microphyllum
Origanum onites
Origanum scabrum
Origanum syriacum
Origanum vulgare
Plectranthus amboinicus
Thymus glandulosus
Lavandula multifida
Origanum minutiflorum
Satureja thymbra

Active ingredients found in Thymus Capitatus

| No | RI | Compound | % |
|---|---|---|---|
| 1 | 935 | α-Thujene | 0.54 |
| 2 | 940 | α-Pinene | 0.38 |
| 3 | 991 | Myrcene | 0.87 |
| 4 | 1019 | α.-αterpinene | 1.11 |
| 5 | 1025 | p-Cymene | 6.25 |
| 6 | 1063 | γ-Terpinene | 6.75 |
| 7 | 1089 | α-terpinolene | 0.26 |
| 8 | 1101 | Linalool | 1.51 |
| 9 | 1179 | Terpinen-4-ol | 1.40 |
| 10 | 1185 | 4-Carvomenthenol | 0.94 |
| 11 | 1260 | Geraniol | 0.25 |
| 12 | 1309 | Carvacrol | 65.38 |
| 13 | 1310 | Thymol | 1.35 |
| 14 | 1358 | Eugenol | 0.21 |
| 15 | 1408 | Carvacryl Acetate | 0.45 |
| 16 | 1427 | β-Caryophyllene | 4.94 |
| 17 | 1461 | α-Humulene | 0.10 |
| 18 | 1487 | allo-aromadendrene | 0.18 |
| 19 | 1685 | α-Bisabolol | 0.35 |
| 20 | 1774 | α-Bisabolol oxide A | 0.11 |
| 21 | 1815 | Hexadecanal | 0.14 |
| 22 | 1870 | 1-Hexadecanol | 0.46 |
| 23 | 1879 | 1-Hexadecanol | 0.13 |
| 24 | 1894 | Rimuene | 0.28 |
| 25 | 1957 | Hexadecanoic acid | 0.68 |
| | | Total identified | 95.02 |
| | | Unknown | 4.98 |

Additional plants contemplated herein are of the genus Thymus.

The genus Thymus (/‘ [symbol] /TY- [symbol] ; thymes) contains about 350 species of aromatic perennial herbaceous plants and subshrubs to 40 cm tall in the family Lamiaceae, native to temperate regions in Europe, North Africa and Asia.

Stems tend to be narrow or even wiry; leaves are evergreen in most species, arranged in opposite pairs, oval, entire, and small, 4-20 mm long, and usually aromatic. Thyme flowers are in dense terminal heads with an uneven calyx, with the upper lip three-lobed, and are yellow, white, or purple.

Several members of the genus are cultivated as culinary herbs or ornamentals, when they are also called thyme after its best-known species, Thymus vulgaris or common thyme.

About 350 species, including:
Thymus adamovicii
Thymus altaicus
Thymus amurensis
Thymus boissieri
Thymus bracteosus
Thymus broussonetii
Thymus caespititius
Thymus camphoratus
Thymus capitatus
Thymus capitellatus
Thymus camphoratus
Thymus carnosus
Thymus cephalotus
Thymus cherlerioides
Thymus ciliatus
Thymus cilicicus
Thymus cimicinus
Thymus citriodorus (Thymus x citriodorus) syn. T. fragrantissimus, T. serpyllum citratus, T. serpyllum citriodorum.^{[7]}—citrus thyme
Thymus comosus
Thymus comptus
Thymus curtus
Thymus decussatus
Thymus disjunctus
Thymus doerfleri
Thymus glabrescens
Thymus herba-barona
Thymus hirsutus
Thymus hyemalis
Thymus inaequalis
Thymus integer
Thymus lanuginosus, syn. T. serpyllum—woolly thyme
Thymus leucospermus
Thymus leucotrichus
Thymus longicaulis
Thymus longiflorus
Thymus mandschuricus
Thymus marschallianus
Thymus mastichina
Thymus membranaceus
Thymus mongolicus
Thymus moroderi
Thymus nervulosus
Thymus nummularis
Thymus odoratissimus
Thymus pallasianus
Thymus pallidus
Thymus pannonicus
Thymus praecox—creeping thyme
Thymus proximus
Thymus pseudolanuginosus, syn. T. serpyllum—woolly thyme
Thymus pulegioides—lemon thyme^{[8]}
Thymus quinquecostatus
Thymus richardii
Thymus satureioides
Thymus serpyllum
Thymus sibthorpii
Thymus striatus
Thymus thracicus—lavender thyme
Thymus villosus
Thymus vulgaris—common thyme
Thymus zygis

List of plants that contain thymol include, but are not limited to:
Euphrasia rostkoviana
Lagoecia cuminoides
Monarda didyma
Monarda fistulosa
Mosla chinensis, Xiang Ru
Origanum compactum
Origanum dictamnus
Origanum onites
Origanum vulgare
Satureja thymbra
Thymus glandulosus
Thymus hyemalis
Thymus vulgaris
Thymus zygis
Trachyspermum ammi

Active ingredients in Thymus vulgaris:

| | | | |
|---|---|---|---|
| | Plant | Low | High |
| Chemical | part | ppm | ppm |

| | | |
|---|---|---|
| 1-OCTEN-3-OL | Shoot | 150 |
| | Shoot | 65 |
| | Shoot | 80 |
| 2,5-DIETHYL-TETRAHYDROFURAN | Shoot | 0 |
| | Shoot | 6 |
| | Shoot | 6 |
| 3-OCTANOL | Shoot | 110 |
| | Shoot | 12 |
| | Shoot | 30 |
| ALPHA-GUAIENE | Shoot | 0.1 |
| | Shoot | 6 |
| | Shoot | 0 |
| ALPHA-HUMULENE | Shoot | 45 |
| | Shoot | 20 |
| | Shoot | 55 |
| ALPHA-PHELLANDRENE | Shoot | 0 |
| | Shoot | 40 |
| | Shoot | 12 |
| ALPHA-PINENE | Shoot | 0 |
| | Shoot | 265 |
| | Shoot | 325 |
| ALPHA-TERPINENE | Shoot | 840 |
| | Shoot | 990 |
| | Shoot | 990 |
| ALPHA-TERPINEOL | Shoot | 55 |
| | Shoot | 55 |
| | Shoot | 25 |
| ALPHA-THUJENE | Shoot | 320 |
| | Shoot | 0 |
| | Shoot | 0 |
| BETA-CARYOPHYLLENE | Shoot | 175 |
| | Shoot | 185 |
| | Shoot | 200 |
| BETA-GUAIENE | Shoot | 0.1 |
| | Shoot | 0 |
| | Shoot | 3 |
| BETA-PHELLANDRENE | Shoot | 80 |
| | Shoot | 60 |
| BETA-PHELLLANDRENE | Shoot | 70 |
| BETA-PINENE | Shoot | 30 |
| | Shoot | 30 |
| | Shoot | 560 |
| BORNEOL | Shoot | 55 |
| | Shoot | 30 |
| | Shoot | 15 |
| CAMPHENE | Shoot | 30 |
| | Shoot | 25 |
| | Shoot | 40 |
| CAMPHOR | Shoot | 0 |
| | Shoot | 0.1 |
| | Shoot | 0 |
| CARVACROL | Shoot | 1,285 |
| | Shoot | 24,850 |
| | Shoot | 23,765 |
| CARVONE | Shoot | 15 |
| | Shoot | 20 |
| | Shoot | 0.1 |
| CARYOPHYLLENE-OXIDE | Shoot | 75 |
| | Shoot | 55 |
| | Shoot | 45 |
| CIS-CARVEOL | Shoot | 0 |
| | Shoot | 0 |
| | Shoot | 3 |
| CIS-SABINENE-HYDRATE | Shoot | 20 |
| | Shoot | 0 |
| | Shoot | 55 |
| CITRONELLOL | Shoot | 12 |
| | Shoot | 0.1 |
| | Shoot | 0 |
| CITRONELLOL-BUTYRATE | Shoot | 0 |
| CITRONELLYL-BUTYRATE | Shoot | 0 |
| | Shoot | 15 |
| DIHYDROCARVONE | Shoot | 0 |
| | Shoot | 0 |
| | Shoot | 12 |
| EHTYL-CINNAMATE | Shoot | 0 |
| EO | Shoot | 31,000 |
| | Shoot | 31,000 |
| | Shoot | 31,000 |
| ETHYL-CINNAMATE | Shoot | 0 |
| | Shoot | 30 |
| GAMMA-TERPINENE | Shoot | 2,700 |
| | Shoot | 1,015 |
| | Shoot | 240 |
| GERANIOL | Shoot | 0 |
| | Shoot | 0 |
| | Shoot | 65 |
| GERANYL-ACETATE | Shoot | 0 |
| | Shoot | 0 |
| | Shoot | 15 |
| GERANYL-BUTYRATE | Shoot | 0 |
| | Shoot | 20 |
| | Shoot | 0 |
| GERANYL-HEXANOATE | Shoot | 0 |
| | Shoot | 0 |
| | Shoot | 6 |
| GERANYL-PROPIONATE | Shoot | 0 |
| | Shoot | 0 |
| | Shoot | 70 |
| GERMACRENE-D | Shoot | 0 |
| | Shoot | 0 |
| | Shoot | 50 |
| LIMONENE | Shoot | 110 |
| | Shoot | 55 |
| | Shoot | 90 |
| LINALOL | Shoot | 35 |
| | Shoot | 55 |
| | Shoot | 25 |
| METHYL-2-METHYL-BUTYRATE | Shoot | 6 |
| | Shoot | 12 |
| | Shoot | 9 |
| MYRCENE | Shoot | 750 |
| | Shoot | 565 |
| | Shoot | 0.1 |
| P-CYMENE | Shoot | 4,445 |
| | Shoot | 1,880 |
| | Shoot | 3,135 |
| TERPINEN-1-OL | Shoot | 6 |
| | Shoot | 15 |
| | Shoot | 0 |
| TERPINEN-4-OL | Shoot | 435 |
| | Shoot | 315 |
| | Shoot | 335 |
| TERPINOLENE | Shoot | 0 |
| | Shoot | 0.1 |
| | Shoot | 45 |
| THYMOL | Shoot | 18,560 |
| | Shoot | 385 |
| | Shoot | 280 |
| TRANS-BERGAMOTENE | Shoot | 9 |
| | Shoot | 9 |
| TRANS-BERGAMOTTENE | Shoot | 9 |
| TRANS-SABINENE-HYDRATE | Shoot | 25 |
| | Shoot | 120 |
| | Shoot | 0 |
| TRICYCLENE | Shoot | 0 |
| | Shoot | 0 |
| | Shoot | 3 |

Active ingredients on the EO of Thymus vulgaris according to some embodiments of the invention, include, but are not limited to:

| No. | RT (min) | Area % of total | Constituents* |
|---|---|---|---|
| 1 | 5.39 | 1.06 | alpha-Thujene |
| 2 | 5.63 | 1.07 | alpha-Pinene |
| 3 | 6.89 | 0.37 | beta-Pinene |
| 4 | 6.97 | 1.53 | beta-Myrcene |
| 5 | 7.53 | 0.33 | alpha-Phellandrene |
| 6 | 7.77 | 3.76 | Carene<δ-2-> |
| 7 | 8.04 | 0.29 | D-Limonene |
| 8 | 8.26 | 0.21 | beta-Phellandrene |
| 9 | 8.46 | 8.41 | para-Cymene |
| 10 | 8.96 | 30.90 | gamma-Terpinene |
| 11 | 9.48 | 0.47 | Terpineol |
| 12 | 12.55 | 0.46 | Terpinen-4-ol |
| 13 | 16.17 | 47.59 | Thymol |
| 14 | 17.32 | 2.68 | Caryophyllene |
| 15 | 19.03 | 0.78 | Cyclohexene, 1-methyl-4-(5- |
| | | | methyl-1-methylene-4-hexeny]) |
| Total | | 99.91% | |

Active Ingredients of Satujera thymbra:

Air dried aerial parts from S. thymbra were collected in Lebanon at random during April 2009. For 3 h the plant material was submitted to steam distillation using a clevenger-type apparatus to produce the essential oil with a yield of 0.84% (w/w). Oil was dried using anhydrous magnesium sulfate and stored at 4° C. S. thymbra oil are analyzed by GC/MS. Nineteen compounds representing 98.8% of the oil sample are identified. The major components of Satureja thymbra L. oil are γ-terpinene (34.06%), carvacrol (23.07%) and thymol (18.82%). Also abundant are ρ-cymene (7.58%), caryophyllene (3.96%), α-terpinene (3.53%) and myrcene (1.70%).

Also contemplated herein are plants of the genus Satujera.

Satureja is a genus of aromatic plants of the family Lamiaceae, related to rosemary and thyme. It is native to North Africa, southern and southeastern Europe, the Middle East, and Central Asia. A few New World species were formerly included in Satureja, but they have all been moved to other genera. Several species are cultivated as culinary herbs called savory, and they have become established in the wild in a few places.

Examples include, but are not limited to:
Satureja adamovicii Šilic—Balkans
Satureja aintabensis P. H. Davis—Turkey
Satureja amani P. H. Davis—Turkey
Satureja atropatana Bunge—Iran
Satureja avromanica Maroofi—Iran
Satureja bachtiarica Bunge—Iran
Satureja boissieri Hausskn. ex Boiss.—Turkey, Iran
Satureja bzybica Woronow—Caucasus
Satureja x caroli-paui G. López—Spain (S. innota x S. montana)
Satureja cilicica P. H. Davis—Turkey
Satureja coerulea Janka—Bulgaria, Romania, Turkey
Satureja cuneifolia Ten—Spain, Italy, Greece, Albania, Yugoslavia, Iraq
Satureja x delpozoi Sánchez-Gómez, J. F. Jiménez & R. Morales—Spain (S. cuneifolia x S. intricata var. gracilis)
Satureja edmondii Briq.—Iran
Satureja x exspectata G. López—Spain (S. intricata var. gracilis x S. montana)
Satureja fukarekii Šilic—Yugoslavia
Satureja hellenica Halácsy—Greece
Satureja hortensis L.
Satureja horvatii Šilic—Greece, Yugoslavia
Satureja icarica P. H. Davis—Greek Islands
Satureja innota (Pau) Font Quer—Spain
Satureja intermedia C. A. Mey.—Iran, Caucasus
Satureja intricata Lange—Spain
Satureja isophylla Rech. f.—Iran
Satureja kallarica Jamzad—Iran
Satureja kermanshahensis Jamzad—Iran
Satureja khuzistanica Jamzad—Iran
Satureja kitaibelii Wierzb. ex Heuff.—Bulgaria, Romania, Yugoslavia
Satureja laxiflora K. Koch—Iran, Iraq, Turkey, Caucasus
Satureja linearifolia (Brullo & Furnari) Greuter—Cyrenaica region of Libya
Satureja macrantha C. A. Mey.—Iran, Iraq, Turkey, Caucasus
Satureja metastasiantha Rech. f.—Iraq
Satureja montana L.—winter savory—southern Europe, Turkey, Syria
Satureja mutica Fisch. & C. A. Mey.—Caucasus, Iran, Turkmenistan
Satureja nabateorum Danin & Hedge—Jordan
Satureja x orjenii Šilic—Yugoslavia (S. horvatii x S. montana)
Satureja pallaryi J. Thiébaut—Syria
Satureja parnassica Heldr. & Sart. ex Boiss.—Greece, Turkey
Satureja pilosa Velen.—Italy, Greece, Bulgaria
Satureja rumelica” Velen.—Bulgaria
Satureja sahendica Bornm.—Iran
Satureja salzmannii (Kuntze) P. W. Ball—Morocco, Spain
Satureja spicigera (K. Koch) Boiss.—Turkey, Iran, Caucasus
Satureja spinosa L.—Turkey, Greek Islands including Crete
Satureja subspicata Bartl. ex Vis.—Austria, Yugoslavia, Albania, Bulgaria, Italy
Satureja taurica Velen.—Crimea
Satureja thymbra L.—Libya, southeastern Europe from Sardinia to Turkey; Cyprus, Lebanon, Palestine
Satureja thymbrifolia Hedge & Feinbrun—Israel, Saudi Arabia
Satureja visianii Šilic.—Yugoslavia
Satureja wiedemanniana (Avé-Lall.) Velen.—Turkey

Active ingredients of Thymbra spicata:

| Compounds | % | RI¹ | RT² |
|---|---|---|---|
| α-pinene | 0.56 | 1028 | 3.64 |
| α-phellandrene | 0.64 | 1033 | 3.71 |
| camphene | 0.06 | 1073 | 4.36 |
| β-pinene | 0.10 | 1113 | 5.16 |
| δ-3-carene | 0.05 | 1155 | 6.10 |
| β-myrcene | 1.04 | 1170 | 6.51 |
| α-terpinene | 1.48 | 1184 | 6.90 |
| dl-limonene | 0.17 | 1202 | 7.43 |
| β-phellandrene | 0.12 | 1212 | 7.69 |
| γ-terpinene | 10.73 | 1252 | 8.86 |
| p-cymene | 12.18 | 1276 | 9.69 |
| α-terpinolene | 0.05 | 1286 | 10.04 |
| oct-1-en-3-ol | 0.11 | 1454 | 16.17 |
| trans sabinene hydrate | 0.05 | 1465 | 16.59 |
| cis sabinene hydrate | 0.03 | 1547 | 19.73 |
| linalool | 0.03 | 1551 | 19.91 |
| trans caryophyllene | 1.28 | 1589 | 21.39 |
| 4-terpineol | 0.53 | 1598 | 21.79 |
| isoborneol | 0.21 | 1694 | 25.36 |
| d-carvone | 0.02 | 1728 | 26.55 |
| anethole | 0.04 | 1826 | 30.05 |
| caryophyllene oxide | 0.65 | 1968 | 34.87 |
| spathulenol | 0.15 | 2125 | 39.56 |
| thymol | 2.77 | 2218 | 41.80 |
| carvacro | 66.86 | 2239 | 42.61 |
| naphthalene³ | 0.08 | 2281 | 44.26 |

¹RT—retention time;
²RI—retention index;
³naphthalene, 1,2,3,4,4a,5,6,7-octahydro-4a-methyl

Also contemplated herein are plants of the genus Thymbra.

Thymbra, common name Mediterranean thyme, is a genus of plants in the family Lamiaceae. As currently categorized, the genus has seven species and one subspecies. It is native to the Mediterranean region of southern Europe, North Africa, and the Middle East.

Examples include, but are not limited to:
Thymbra calostachya (Rech. f.) Rech. f.—Crete
Thymbra capitata (L.) Cav.—widespread from Morocco+Portugal to Turkey+Palestine
Thymbra sintenisii Bornm. & Azn.—Iraq, Turkey
Thymbra spicata L.—Greece, Turkey, Syria, Lebanon, Palestine, Israel, Iraq, Iran
Thymbra thymbrifolia (Hedge & Feinbrun) Bräuchler, comb. nov.—Israel, Palestine, Judean Desert, Khirbet el Mird
Thymbra nabateorum (Danin & Hedge) Bräuchler, comb. nov.—W of Jordan and the adjacent N of Saudi Arabia
Thymbra linearifolia (Brullo & Furnari) Bräuchler, comb. nov.—Libya

Chemical Composition of Rhus coriaria (Sumac)

Characterization and identification of chemical compounds of Sumac using HPLC-MS method identified 191 compounds in Rhus coriaria and classified them as generally being:
78 hydrolysable tannins (e.g., gallotannins, e.g., penta, hexa, hepta, octa, nona and decagalloyl-glucoside)
59 flavonoids (e.g., Quercetin, Myrecetin 3-rhamnoside and Quercetin 3-glucoside)
9 anthocyanins (e.g., Delphidin-3-glucoside, Cyanidin 3-(2″-galloyl)galactoside, Cyanidin-3-glucoside, 7-methyl-cyanidin-3-(2″galloyl)galactoside, 7-methyl-cyanidin-3-galactoside)
2 isoflavonoids
2 terpenoids
1 diterpene
38 other unidentified compounds.

According to specific embodiments, the phenolic compounds in Sumac are the compounds that constitute its phytochemical activity along with anthocyanins. The most abundant phenolic compound in sumac fruits was found to be Gallic acid.

Hydrolysable tannins compose the highest percentage in the Sumac fruits, followed by flavonoids. This emphasizes the antioxidant potential of the fruit, a plant part contemplated herein as a specific embodiment. Following hydrolysable tannins, comprising almost 20% of the fruit's mass, are other unidentified compounds. Subsequently there are anthocyanins, isoflavonoids, terpenoids and diterpenes. The chemical properties of sumac fruit is conducted on ripe fruits and have found a 2.6% protein content, 7.4% fat content, 14.6% fiber content, 1.8% ash. Also, a calorimetric calculation showed that 100 g of sumac fruit contains 147.8 kcal.

Hydrolysable tannins compose the highest percentage in the Sumac fruits, followed by flavonoids. This emphasizes the antioxidant potential of the fruit. Following hydrolysable tannins, comprising almost 20% of the fruit's mass, are other unidentified compounds. Subsequently there are anthocyanins, isoflavonoids, terpenoids and diterpenes. The chemical properties of sumac fruit is conducted on ripe fruits and have found a 2.6% protein content, 7.4% fat content, 14.6% fiber content, 1.8% ash. Also, a calorimetric calculation showed that 100 g of sumac fruit contains 147.8 kcal.

Other active ingredients or any combinations thereof include, but are not limited to, methyla gallate, gathisflavone, sumaflavone, hinfikflavone, photocatechuic acid, penta-galloylglucose, hinokiflavone, β-caryophyllene, Delphidin-3-glucoside, Cyanidin 3-(2″-galloyl)galactoside, Cyanidin-3-glucoside, 7-methyl-cyanidin-3-(2″galloyl)galactoside, 7-methyl-cyanidin-3-galactoside, quercetin-3-glucoside, kampferol, myricetin, butein, D-limonine.

According to a specific embodiment, the active ingredient or combination thereof includes a volatile compound, e.g., terpene hydrocarbons, monoterpene and sesquiterpene hydrocarbons, specifically β-caryophyllene and α-pinene, Coririanaphthyl ether, Coriarioic acid and Coriariacthracenyl ester.

According to a specific embodiment, the active ingredient or combination thereof includes a fatty acid, e.g., oleic acid, linoleic acid, palmitic acid, β-caryophillene, cembrene stearic acid, Myristic acid, α-linolenic acid.

According to a specific embodiment, the active ingredient or combination thereof includes a mineral, e.g., potassium, calcium, magnesium, phosphorus, aluminum, iron, sodium, boron, zinc, cadmium, selenium.

According to a specific embodiment, the active ingredient or combination thereof includes a vitamin, e.g., thiamin B₁, riboflavin B₂, pyridoxine B₆, cyanocobalamin B₁₂, nicotinamide, biotin and ascorbic acid.

According to a specific embodiment, a methanol or ethanol extract is performed, e.g., ethanol concentration is 80%; extraction time is 1 h; extraction temperature is 40° C.; particle size 1.0 mm; and solvent to sumac ratios 15:1 ml/g. Other extraction procedures include, but are not limited to, those described in Sakhr and Khatib Heliyon. 2020 January; 6(1): e03207, which is hereby incorporated by reference in its entirety.

According to another embodiment, the plant part is leaf.

Also contemplated herein are plants of the genus Rhus.

Examples include, but are not limited to:

### Asia and Southern Europe

Rhus chinensis Mill.—Chinese sumac
Rhus coriaria—Tanner's sumac
Rhus delavayi Franchet

### Australia, Pacific

Rhus taitensis Guill. (Northeast Australia, Malesia, Micronesia, French Polynesia)
Rhus sandwicensis A. Gray—neneleau (Hawaii)

### North America

Rhus aromatica—fragrant sumac
Rhus copallinum—winged or shining sumac
Rhus glabra—smooth sumac
Rhus integrifolia—lemonade sumac
Rhus kearneyi—Kearney sumac
Rhus lanceolata—prairie sumac
†Rhus malloryi Wolfe & Wehr—Ypresian, Washington
Rhus michauxii—Michaux's sumac
Rhus microphylla—desert sumac, littleleaf sumac
Rhus ovata—sugar sumac
†Rhus republicensis Flynn, DeVore, & Pigg-Ypresian, Washington
†Rhus rooseae Manchester—Middle Eocene, Oregon
Rhus trilobata Nutt.—skunkbush sumac
Rhus typhina—staghom sumac
Rhus virens Lindh. ex A. Gray—evergreen sumac

Chemical Composition of Panax ginseng (ginseng)

Characterization and identification of chemical compounds of ginseng using a variety of methods identified a large variety of compounds in Panax ginseng and classified them as generally being:
Saponin Glycosides (e.g., ginsenosides)
Phytosterols (e.g. stigmasterol, beta-sterol)
Sesquiterpenes (e.g. beta-alamene and beta-selinine)
Flavenoids (e.g. Kaempferol)
Polyacetylenes (e.g. panaxynol, ginsenoyne A)
Alkaloids (e.g. fumarine, girinimbin)
Polysaccharides
Phenolic compounds (e.g. elemicin, dauricin, maltol).

According to specific embodiments, the saponin compounds in ginseng and the polysaccharide compounds are the compounds that constitute its phytochemical activity. The most abundant saponin compound in ginseng root was found to be ginsenoside. Polysaccharides from ginseng have been identified as NGP, WGP, 1-KGP, 4-KGP, WGPE and EGP, with WGP and WGPE being the most abundant, depending on the species of ginseng plant material used for extraction.

Most ginseng saponins belong to a family of steroids with a four trans-ring rigid steroid skeleton. They are also referred to as ginsenosides, triterpenoid saponins or dammarane derivatives. More than 200 saponins have been isolated from ginseng plants. In addition to ginseng root, saponins have been identified in ginseng leaves and stems, flower buds, fruits, berries, and seeds. Because steaming or heating changes the saponin profile of ginseng products, ginseng saponins have also been identified in the processed root, leaf, flower-bud and berry.

Ginseng saponins are divided into several groups. Two major groups are the protopanaxadiol (PPD)-type saponins with sugar moieties attached to the C-3 and/or C-20 and the protopanaxatriol (PPT) group with sugar moieties at C-6 and/or at C-20. Other groups include the ocotillol-type with a five-membered epoxy ring at C-20, the oleanane-type with a nonsteroidal structure, and the dammarane type with a modified C-20 side chain. As techniques are developed for chemical purification and structural identification, novel ginseng saponins continue to be discovered.

The table below shows ginsenoside compounds recovered from ginseng extracts prepared by different extraction procedures:

GINSENOSIDES

| | Solvent system^{a} | | Obtained | |
|---|---|---|---|---|
| Material | (volume ratio) | Detection^{b} | compound | Isolation efficiency^{c} |
| P. | Hex-n-BuOH-H₂O | TLC | Ginsenosides Rb1, | 157, 13, 56, and 17 mg of |
| notoginseng, | (3:4:7) | | Re, Rg1 and | Rb1, Re, Rg1 and R1 |
| root | | | notoginsenoside R1 | from 283 mg MeOH |
| | | | | extract of five tablets |
| P. ginseng, | CH₂Cl₂—MeOH—NH₄OAc—iPrOH | ELSD | Ginsenosides Rf, Rd, | 10.7, 11.0, 13.4 and 13.9 |
| root | | | Re, and Rb1 | mg of Rf, Rd, Re and |
| | (6:2:4:3) | | | Rb1 from 480 mg |
| | | | | enriched fraction by |
| | | | | macroporous resin |
| P. | CHCl₃—MeOH-2-BuOH—H₂O | ELSD | Ginsenosides Rg1, | Not provided |
| notoginseng, | (5:6:1:4 ) | | Rd, Re, Rb1 and | |
| root | EtOAc-n-BuOH-H₂O | | notoginsenoside R1 | |
| | (1:1:2) | | | |
| Red P. | CH2Cl₂—MeOH—H₂O—iPrOH | ELSD | Ginsenosides Rg3, | 32.2, 26.6, 28.6 and 8.1 |
| ginseng, | (6:6:4:1) | | Rk1, Rg5 and F4 | mg of Rg3, Rk1, Rg5 and |
| steamed root | | | | F4 from 350 mg enriched |
| | | | | fraction by RP- |
| | | | | C₁₈ column |
| P. ginseng, | EtOAc—iPrOH— | UV | Ginsenoside Ro | 61 mg Ro from 100 mg |
| root | 0.1% formic acid | | | enriched sample by |
| | H₂O (3:1:5) | | | normal-phase MPLC |

^{a}Abbreviations: Hex: n-hexane; BuOH: butanol; CH₂Cl₂: methylene chloride; MeOH: methanol; NH₄OAc: ammonium acetate; iPrOH: isopropanol; CHCl₃: chloroform; EtOAc: ethyl acetate.
^{b}Abbreviations: TLC: thin layer chromatography; ELSD: evaporative light scattering detection; UV: ultraviolet.
^{c}Abbreviations: RP: reversed-phase; MPLC: medium-pressure liquid chromatography.

The table below shows the chemical formulae of 123 dammarane-type saponins isolated from various parts of Panax plants. They are placed in the order of the structure type.

Dammarane - type saponin ginsenosides

| No. | Name | Formula | Plant Material |
|---|---|---|---|
| 1 | Floralginsenoside M | C₅₃H₉₀O₂₂ | Flower buds of P. ginseng |
| 2 | Floralginsenoside N | C₅₃H₉₀O₂₂ | Flower buds of P. ginseng |
| 3 | Floralquinquenoside E | C₅₃H₉₀O₂₂ | Flower buds of P. quinquefolius |
| 4 | Ginsenoside Rh5 | C₃₇H₆₄O₉ | Roots and rhizomes of P. |
| | | | vietnamensis |
| 5 | Notoginsenoside FP 1 | C₄₇H₈₀O₁₈ | Fruit pedicels of P. notoginseng |
| 6 | Notoginsenoside M | C₄₈H₈₂O₁₉ | Roots of P. notoginseng |
| 7 | Notoginsenoside N | C₄₈H₈₂O₁₉ | Roots of P. notoginseng |
| 8 | Notoginsenoside Rw1 | C₄₆H₇₈O₁₇ | Rhizomes of P. notoginseng |
| 9 | Notoginsenoside T3 | C₃₈H₆₆O₉ | Acid hydrolysate roots of P. |
| | | | notoginseng |
| 10 | Notoginsenoside U | C₄₂H₇₂O₁₄ | Roots of P. notoginseng |
| 11 | Quinquenoside L17 | C₄₇H₈₀O₁₈ | Leaves and stems of P. quinquefolius |
| 12 | Yesanchinoside D | C₄₄H₇₄O₁₅ | Underground part of P. japonicus |
| 13 | Yesanchinoside E | C₅₄H₉₂O₂₃ | Underground part of P. japonicus |
| 14 | Yesanchinoside F | C₅₆H₉₄O₂₄ | Underground part of P. japonicus |
| 15 | 20(S)-acetylated Rg2 | C₄₄H₇₄O₁₄ | Roots of P. quinquefolius |
| 16 | 20(R)-acetylated Rg2 | C₄₄H₇₄O₁₄ | Roots of P. quinquefolius |
| 17 | Malonylginsenoside Ra3 | C₆₂H₁₀₂O₃₀ | Fresh roots of P. ginseng |
| 18 | Malonylnotoginsenoside R4 | C₆₂H₁₀₂O₃₀ | Roots of P. ginseng |
| 19 | Notoginsenoside FP2 | C₅₈H₉₈O₂₆ | Fruit pedicels of P. notoginseng |
| 20 | Notoginsenoside FT1 | C₄₇H₈₀O₁₇ | Acid hydrolysate roots of P. |
| | | | notoginseng |
| 21 | Notoginsenoside L | C₅₃H₉₀O₂₂ | Roots of P. notoginseng |
| 22 | Notoginsenoside O | C₅₂H₈₈O₂₁ | Flower buds of P. notoginseng |
| 23 | Notoginsenoside P | C₅₂H₈₈O₂₁ | Flower buds of P. notoginseng |
| 24 | Notoginsenoside Q | C₆₃H₁₀₆O₃₀ | Flower buds of P. notoginseng |
| 25 | Notoginsenoside S | C₆₃H₁₀₆O₃₀ | Flower buds of P. notoginseng |
| 26 | Notoginsenoside T | C₆₄H₁₀₈O₃₁ | Flower buds of P. notoginseng |
| 27 | Quinquenoside L10 | C₄₇H₈₀O₁₇ | Leaves and stems of P. quinquefolius |
| 28 | Quinquenoside L14 | C₄₇H₈₀O₁₇ | Leaves and stems of P. quinquefolius |
| 29 | Yesanchinoside J | C₆₁H₁₀₂O₂₈ | Underground part of P. japonicus |
| 30 | Floralginsenoside A | C₄₂H₇₂O₁₆ | Flower buds of P. ginseng |
| 31 | Floralginsenoside C | C₄₁H₇₀O₁₅ | Flower buds of P. ginseng |
| 32 | Floralginsenoside H | C₅₀H₈₄O₂₁ | Flower buds of P. ginseng |
| 33 | Floralginsenoside J | C₄₈H₈₂O₂₀ | Flower buds of P. ginseng |
| 34 | Floralginsenoside Ka | C₃₆H₆₂O₁₁ | Flower buds of P. ginseng |
| 35 | Floralginsenoside Tc | C₅₃H₉₀O₂₄ | Flower buds of P. ginseng |
| 36 | Floralquinquenoside B | C₄₂H₇₂O₁₅ | Flower buds of P. quinquefolius |
| 37 | Floralquinquenoside D | C₄₂H₇₂O₁₅ | Flower buds of P. quinquefolius |
| 38 | Floranotoginsenoside B | C₅₃H₉₀O₂₄ | Flowers of P. notoginseng |
| 39 | Floranotoginsenoside C | C₅₃H₉₀O₂₄ | Flowers of P. notoginseng |
| 40 | Ginsenoside I | C₄₈H₈₂O₂₀ | Flower buds of P. ginseng |
| 41 | Ginsenoside II | C₄₈H₈₂O₂₀ | Flower buds of P. ginseng |
| 42 | Ginsenoside SL1 | C₃₆H₆₂O₁₁ | Steamed leaves of P. ginseng |
| 43 | Floralginsenoside B | C₄₂H₇₂O₁₆ | Flower buds of P. ginseng |
| 44 | Floralginsenoside D | C₄₁H₇₀O₁₅ | Flower buds of P. ginseng |
| 45 | Floralginsenoside E | C₄₂H₇₂O₁₅ | Flower buds of P. ginseng |
| 46 | Floralginsenoside F | C₄₂H₇₂O₁₅ | Flower buds of P. ginseng |
| 47 | Floralginsenoside G | C₅₀H₈₄O₂₁ | Flower buds of P. ginseng |
| 48 | Floralginsenoside I | C₄₈H₈₂O₂₀ | Flower buds of P. ginseng |
| 49 | Floralginsenoside K | C₄₈H₈₂O₂₁ | Flower buds of P. ginseng |
| 50 | Floralginsenoside O | C₅₃H₉₀O₂₄ | Flower buds of P. ginseng |
| 51 | Floralginsenoside P | C₅₃H₉₀O₂₃ | Flower buds of P. ginseng |
| 52 | Floralquinquenoside A | C₃₆H₆₂O₁₁ | Flower buds of P. quinquefolius |
| 53 | Floralquinquenoside C | C₄₂H₇₂O₁₅ | Flower buds of P. quinquefolius |
| 54 | Ginsenoside Rh6 | C₃₆H₆₂O₁₁ | Leaves of P. ginseng |
| 55 | Floralginsenoside La | C₄₈H₈₂O₁₉ | Flower buds of P. ginseng |
| 56 | Floralginsenoside Lb | C₄₈H₈₂O₁₉ | Flower buds of P. ginseng |
| 57 | Floranotoginsenoside D | C₅₃H₉₀O₂₃ | Flowers of P. notoginseng |
| 58 | Ginsenoside Rg7 | C₃₆H₆₀O₉ | Leaves of P. ginseng |
| 59 | Notopanaxoside A | C₃₆H₆₂O₁₀ | Roots of P. notoginseng |
| 60 | Notoginsenoside FT3 | C₄₇H₈₀O₁₈ | Acid hydrolysate roots of P. |
| | | | notoginseng |
| 61 | Floranotoginsenoside A | C₅₃H₉₀O₂₃ | Flowers of P. notoginseng |
| 62 | Ginsenoside ST2 | C₃₆H₆₂O₁₀ | Steamed leaves of P. ginseng |
| 63 | Notoginsenoside Rw2 | C₄₁H₇₀O₁₄ | Rhizomes of P. notoginseng |
| 64 | Notoginsenoside ST5 | C₄₇H₈₀O₁₈ | Steamed roots of P. notoginseng |
| 65 | Yesanchinoside H | C₅₃H₉₀O₂₃ | Underground part of P. japonicus |
| 66 | Ginsenoside Ki | C₃₆H₆₂O₁₀ | Leaves of P. ginseng |
| 67 | Ginsenoside Km | C₃₆H₆₂O₁₀ | Leaves of P. ginseng |
| 68 | Quinquenoside L2 | C₄₈H₈₂O₁₉ | Leaves and stems of P. quinquefolius |
| 69 | Dammar-25(26)-ene-3,6,12,20,22,24- | C₃₀H₅₂O₆ | Leaves of P. ginseng |
| | hexanol | | |
| 70 | Floralginsenoside Kb | C₄₅H₇₆O₁₉ | Flower buds of P. ginseng |
| 71 | Floralginsenoside Kc | C₄₅H₇₆O₂₀ | Flower buds of P. ginseng |
| 72 | Floralginsenoside Ta | C₃₆H₆₀O₁₀ | Flower buds of P. ginseng |
| 73 | Vina-ginsenoside R25 | C₄₂H₇₀O₁₅ | Roots and rhizomes of P. |
| | | | vietnamensis |
| 74 | Floralginsenoside Tb | C₃₅H₆₂O₁₁ | Flower buds of P. ginseng |
| 75 | Quinquenoside L9 | C₄₂H₇₄O₁₅ | Leaves and stems of P. quinquefolius |
| 76 | Quinquenoside L16 | C₅₄H₉₄O₂₅ | Leaves and stems of P. quinquefolius |
| 77 | 25-OCH₃-PPD | C₃₁H₅₆O₄ | Leaves of P. notoginseng |
| 78 | 25-OH-PPD | C₃₀H₅₄O₄ | Fruits of P. ginseng |
| 79 | 25-OH-PPT | C₃₀H₅₄O₅ | Fruits of P. ginseng |
| 80 | Notoginsenoside FT2 | C₄₇H₈₂O₁₈ | Acid hydrolysate roots of P. |
| | | | notoginseng |
| 81 | Notoginsenoside T4 | C₃₆H₆₂O₁₁ | Acid hydrolysate roots of P. |
| | | | notoginseng |
| 82 | Quinquenoside L1 | C₄₈H₈₀O₁₈ | Leaves and stems of P. quinquefolius |
| 83 | Quinquefoloside La | C₅₄H₉₂O₂₃ | Leaves of P. quinquefolius |
| 84 | Quinquefoloside Lc | C₅₄H₉₂O₂₃ | Leaves of P. quinquefolius |
| 85 | Dammar-(E)-20(22)-ene-3,12,25-triol | C₃₀H₅₂O₃ | Acid hydrolysate roots of P. ginseng |
| 86 | Notoginsenoside ST1 | C₃₆H₆₂O₁₀ | Steamed roots of P. notoginseng |
| 87 | Ginse noside Rg6 | C₄₂H₇₀O₁₂ | Stem-leaves of P. ginseng |
| 88 | Ginsenoside Rs4 | C₄₂H₇₀O₁₂ | Steamed roots of P. notoginseng |
| 89 | Ginsenoside Rs6 | C₄₂H₇₀O₁₂ | Steamed roots of P. notoginseng |
| 90 | Isoginsenoside Rh3 | C₃₆H₆₀O₇ | Fruits of P. ginseng |
| 91 | Ginsenoside Rh5 | C₃₆H₆₀O₉ | Leaves of P. ginseng |
| 92 | Ginsenoside SL2 | C₄₂H₇₀O₁₄ | Steamed leaves of P. ginseng |
| 93 | Ginsenoside ST1 | C₃₆H₆₀O₁₀ | Steamed leaves of P. ginseng |
| 94 | Notoginsenoside ST2 | C₄₃H₇₄O₁₅ | Steamed roots of P. notoginseng |
| 95 | Notoginsenoside ST3 | C₄₃H₇₄O₁₅ | Steamed roots of P. notoginseng |
| 96 | Ginsenoside Rg8 | C₄₂H₇₀O₁₂ | Roots of P. quinquefolius |
| 97 | Notoginsenoside T1 | C₃₆H₆₀O₁₀ | Acid hydrolysate roots of P. |
| | | | notoginseng |
| 98 | Notoginsenoside T2 | C₃₆H₆₂O₁₀ | Acid hydrolysate roots of P. |
| | | | notoginseng |
| 99 | Ginsenoside Rg1-12,23-epoxy | C₄₂H₇₀O₁₄ | Leaves of P. ginseng |
| 100 | Ginsenoside Rh9 | C₃₆H₆₀O₉ | Leaves of P. ginseng |
| 101 | Quinquefoloside-Lb | C₅₃H₈₈O₂₂ | Leaves of P. quinquefolius |
| 102 | Ginsenoside Rkl | C₄₂H₇₀O₁₂ | Processed roots of P. ginseng |
| 103 | Ginsenoside Rk2 | C₃₆H₆₀O₇ | Processed roots of P. ginseng |
| 104 | Ginsenoside Rk3 | C₃₆H₆₀O₈ | Processed roots of P. ginseng |
| 105 | Ginsenoside Rs5 | C₃₈H₆₂O₉ | Steamed roots of P. notoginseng |
| 106 | Ginsenoside Rs7 | C₃₈H₆₂O₉ | Steamed roots of P. notoginseng |
| 107 | Notoginsenoside T5 | C₄₁H₆₈O₁₂ | Acid hydrolysate roots of P. |
| | | | notoginseng |
| 108 | Ginsenoside Rzl | C₄₂H₇₀O₁₂ | Steamed roots of P. notoginseng |
| 109 | Ginsenoside SL3 | C₄₂H₇₀O₁₄ | Steamed leaves of P. ginseng |
| 110 | Ginsenoside Rh8 | C₃₆H₆₀O₉ | Leaves of P. ginseng |
| 111 | Ginsenoside Rh7 | C₃₆H₆₀O₉ | Leaves of P. ginseng |
| 112 | Yesanchinoside G | C₅₃H₈₈O₂₃ | Underground part of P. japonicus |
| 113 | Yesanchinoside I | C₅₉H₁₀₀O₂₆ | Underground part of P. japonicus |
| 114 | Hexanordammaran | C₂₄H₄₀O₄ | Leaves of P. ginseng |
| 115 | Notoginsenoside R10 | C₃₀H₅₀O₉ | Steamed leaves of P. ginseng |
| 116 | Yesanchinoside A | C₄₄H₇₄O₁₆ | Underground part of P. japonicus |
| 117 | Yesanchinoside B | C₄₈H₈₂O₂₀ | Underground part of P. japonicus |
| 118 | Yesanchinoside C | C₄₇H₈₀O₁₉ | Underground part of P. japonicus |
| 119 | Panaxadione | C₃₀H₄₈O₅ | Seeds of P. ginseng |
| 120 | Polyacetyleneginsenoside Ro | C₆₅H₁₀₀O₂₁ | Roots of P. ginseng |
| 121 | Isodehydroprotopanaxatriol | C₃₀H₅₀O₃ | Acid hydrolysate roots of P. ginseng |
| 122 | 20,25-epoxy-dammaran-2-en-6,12-diol | C₃₀H₅₀O₃ | Acid hydrolysate roots of P. ginseng |
| 123 | 3-methyl-28-nordammaran-2-en-6,12-diol | C₃₀H₅₀O₃ | Acid hydrolysate roots of P. ginseng |

Analysis of ginseng root (Japanese ginseng) has indicated (per 100 grams root) 0.17 g (0.17%) total fat, 50 mg sodium, 8.82 g (8.82%) total carbohydrates comprising 2.3 g dietary fiber and 3.85 g sugars and 0.71 g (0.71%) protein content. Calorimetric calculation showed that 100 g of ginseng root contains 37 kcal.

According to a specific embodiment, the active ingredient or combination thereof includes a ginsenoside, e.g. a protopanaxadiol (PPD)-type saponin with sugar moieties attached to the C-3 and/or C-20, a protopanaxatriol (PPT) saponin with sugar moieties at C-6 and/or at C-20, an ocotillol-type saponin with a five-membered epoxy ring at C-20, an oleanane-type saponin with a nonsteroidal structure, and a dammarane type saponin. Some specific ginsenosides include, but are not limited to notoginsenosides, yesanchinosides, panaxodione, floralginsenosides and ginsenosides Rg1, Rd, Re, Rb1, R1, Rg3, Rk1, Rf, Rg5, F4, Ro.

According to a specific embodiment, the active ingredient or combination thereof includes a volatile compound, e.g., terpene hydrocarbons, monoterpene and sesquiterpene hydrocarbons, specifically β-alamene and β-selenine.

According to a specific embodiment, the active ingredient or combination thereof includes a phytosterol, e.g., stigmasterol, beta-sterol.

According to a specific embodiment, the active ingredient or combination thereof includes a polyacetylene, e.g., panaxynol, ginsenoyne A.

According to a specific embodiment, the active ingredient or combination thereof includes a flavenoid, e.g., Kaempferol.

According to a specific embodiment, the active ingredient or combination thereof includes an alkaloid, e.g., fumarine, girinimbin.

According to a specific embodiment, the active ingredient or combination thereof includes a polysaccharide, e.g., WGP, KGP-1, KGP-4, WGPE, NGP, EGP.

According to a specific embodiment, the active ingredient or combination thereof includes a phenolic compound, e.g., elemicin, dauricin, maltol.

According to a specific embodiment, the active ingredient or combination thereof includes a mineral, e.g., potassium, calcium, magnesium, phosphorus, aluminum, iron, sodium, boron, zinc, cadmium, selenium.

According to a specific embodiment, the active ingredient or combination thereof includes a vitamin, e.g., vitamin D, vitamin A and vitamin C.

According to a specific embodiment, a methanol or ethanol extract is performed, e.g., ethanol concentration is 80%; extraction time is 24 h; extraction temperature is 80-90° C.; particle size 1.0 mm; and solvent to ginseng ratio of 20:1 ml/g. Other extraction procedures include, but are not limited to, those described in Dong et al. 2017 Phytother Res Aug; 19(8): 684-688, which is hereby incorporated by reference in its entirety.

According to another embodiment, the plant part is leaf.

Also contemplated herein are plants of the genus Panax.

Examples include, but are not limited to:

| | Common name and |
|---|---|
| | geographical |
| Ginseng Species | designation |
| P. gensing | Korean ginseng |
| P. quinquefolius | American ginseng |
| P. notoginseng | Chinese ginseng |
| P. japonicas | Japanese ginseng |
| P. omiensis | Omei gensing |
| P. pseudoginseng | Himalayan ginseng |
| P. assamicus | N/A |
| P. shangianus | N/A |
| P. sinensis | N/A |
| P. stipuleanatus | Pingpien ginseng |
| P. trifolius | Dwarf ginseng |
| P. variabilis | N/A |
| P. vietnamensis | Vietnamese ginseng |
| P. wangianus | Narrow-leaved |
| P. bipinnatifidus | Feather-leaf bamboo |
| | ginseng |
| P. sokpayensis | N/A |
| P. zingiberensis | Ginger ginseng |

Korean ginseng cultivars suitable for use with the present invention include, but are not limited to: Chunpoong, Yunpoong, Gopoong, Sunpoong, Gumpoong, Cheongsun, Sunhyang, Sunun, Sunone, K-1, G-1 and Kowon. Chinese ginseng cultivars suitable for use with the present invention include, but are not limited to Jilin Huangguo Reshen, Jishen 01, Fuxing 01, Fuxing 02, Kangmei 01, Xinkaihe 01, Xinkaihe 02, Zhongnong Huangfengshen and Zhongda Linxiashen.

Chemical Composition of Boswellia Species (Frankincense, Olibanum)

Olibanum, also known as frankincense, is a natural oleo-gum-resin that exudes from tappings in the bark of Boswellia trees. There are approximately 23 species of trees in the genus Boswellia, which grow mainly in Arabia, on the eastern coast of Africa and in India. Characterization and identification of chemical compounds of Olibanum using a variety of methods identified a large variety of compounds in the gum resin of Boswellia tree species and classified them as generally being:
Alcohol-soluble resins (e.g. diterpenes, triterpenes)
Highly aromatic essential oils (e.g. mono- and sesquiterpenes)
Water soluble gums

According to specific embodiments, Olibanum comprises 65-85% alcohol-soluble resins, about 5-9% highly aromatic essential oils and the remainder water soluble gums.

In India, the main commercial sources of Boswellia serrata are Andhra Pradesh, Gujarat, Madhya Pradesh, Jharkhand and Chhattisgarh. Regionally, it is also known by different names. The botanical origin and vernacular names of Boswellia serrata are given in below Table 1. Salai, an oleo gum-resin, is a plant exudate of genus Boswellia (Family: Burseraceae). It is tapped from the incision made on the trunk of the tree, which is then stored in specially made bamboo basket. The semi-solid gum-resin is allowed to remain in the basket for about a month during which its fluid content locally known as ‘ras’ keeps flowing out. The residue, semi-solid to solid part, is the gum-resin which hardens slowly into amorphous, tear-shaped products with an aromatic scent. Then, it is broken into small pieces by wooden mallet or chopper and during this process all impurities including bark pieces etc. are removed manually. The gum-resin is then graded according to its flavour, colour, shape and size. Generally four grades i.e. Superfine, Grade I, Grade II and Grade III are available in the market. The fresh gum obtained from the tree is hot with pleasant flavour and slightly bitter in taste. It had been the ‘frankincense’ of ancient Egyptians, Greeks and Romans who used it as prized incense, fumigant as well as a multipurpose aromatic. It is generally used in making incense powder and sticks.

TABLE 1
BOTANICAL ORIGIN AND VERNACULAR NAMES
OF BOSWELLIA SERRATA

| Botanical origin | Vernacular names |
|---|---|
| Division: Spermatophyta | English: Indian Ollbanum [symbol] or |
| | Indian frankinsense |
| Sub-division: Anglospermae | Hindi: Kundur, Salai |
| Tribe: Rosopsida | Bengall: Kundur, Salai |
| Sub-tribe: Rosidae S. Lat. | Gujarati [symbol] : Dhup, Gugall |
| Over class: Rutanae | Kannada: Chitta, Guguladhuph |
| Class: Anacardiales | Malayalam [symbol] : Parangl [symbol] , Saambraani |
| Family: Burseraceae | Tamil: Parangi, Saambraani |
| Genus: Boswallia | Telugu: Phirangi, Saambraani |
| Species: serrata | Sanskrit: Ashvamutri, Kundara, Skallaki. |

[symbol]

indicates data missing or illegible when filed

The oleo gum-resins contain 30-60% resin, 5-10% essential oils, which are soluble in the organic solvents, and the rest is made up of polysaccharides (˜65% arabinose, galactose, xylose) which are soluble in water. The resins have a fragrant aroma because of the presence of essential oils and this accounts for their commercial importance.

According to specific embodiments, the common components of Olibanum belonging to the terpene and sesquiterpene families, or their terpenoid derivatives include, but are not limited to α- and β-pinene, α-limonene, myrcene, linalool, α-cubebene, γ-cadinene, β-bourbonene, and α-phellandrene dimer compounds in Olibanum are the compounds that constitute its phytochemical activity. Several oxygenated isoprenoid derivatives have also been identified, such as carbonyl derivatives (e.g., carvone, fenchone) and alcohol-containing terpene and sesquiterpene derivatives (e.g., transpinocarveol, cis-verbenol, and cembrenol), as well as ester-containing compounds (e.g., α-terpinyl acetate and bornyl acetate).

Diverse investigators have reported that limonene is the most abundant volatile in Olibanum, while others have identified octanol acetate, α-pinene and α-thujene as most abundant depending on the species of Boswellia resin used for extraction.

More than 300 essential oils have been isolated from Boswellia ssp.

The table below shows the essential oils recovered from Olibanum extracts prepared by different extraction procedures, from diverse Boswellia ssp.:

| Number | Compound |
|---|---|
| 1 | 5,5-Dimethyl-1-vinylbicyclo-[2.1.1]-hexane |
| 2 | Anethol |
| 3 | Benzyl tiglate |
| 4 | trans-α-Bergamotene |
| 5 | Bornyl acetate |
| 6 | β-Bourbonene |
| 7 | Cadinene |
| 8 | γ-Cadinene |
| 9 | Camphene |
| 10 | Camphor |
| 11 | m-Camphorene |
| 12 | p-Camphorene |
| 13 | Carene-3 |
| 14 | (E)-β-Caryophyllene |
| 15 | Cembrene A |
| 16 | Cembrenol |
| 17 | 1,8 Cineol |
| 18 | Citronellol |
| 19 | α-Copaene |
| 20 | β-Copaene |
| 21 | p-Cymene |
| 22 | m-Cymene |
| 23 | Elemol |
| 24 | Elemicine |
| 25 | epi-Cubenol |
| 26 | Estragol |
| 27 | Eudesmol |
| 28 | 10-epi-γ-Eudesmol |
| 29 | Fenchone |
| 30 | Geraniol |
| 31 | Germacrene D |
| 32 | Humulene epoxide |
| 33 | Isoincensole |
| 34 | Isomenthone |
| 35 | Kessane |
| 36 | Limonene |
| 37 | Linalool |
| 38 | Linalyl acetate |
| 39 | Menthone |
| 40 | Methylchavicol |
| 41 | Methylisoeugenol |
| 42 | Methyleugenol |
| 43 | γ-Muurolene |
| 44 | Myrcene |
| 45 | Neocembrene A |
| 46 | Nerolidol |
| 47 | cis-β-ocimene |
| 48 | (Z)-Ocimene |
| 49 | (E)-β-Ocimene |
| 50 | Perillene |
| 51 | α-Phellandrene |
| 52 | β-Phellandrene |
| 53 | α-Pinene |
| 54 | β-Pinene |
| 55 | trans-Pinocarveol |
| 56 | Sabinene |
| 57 | cis-Sabinol |
| 58 | Terpinin-4-ol |
| 59 | Terpinen-4-ol |
| 60 | Terpinolene |
| 61 | α-Terpineol |
| 62 | α-Terpinene |
| 63 | α-Terpinene |
| 64 | γ-Terpinene |
| 65 | Terpinyl acetate |
| 66 | Terpinyl isobutyrate |
| 67 | Tetrahydrolinalool |
| 68 | α-Thujene |
| 69 | α-Thujone |
| 70 | β-Thujone |
| 71 | Tricyclene |
| 72 | Undecenol |
| 73 | trans-Verbenol |
| 74 | β-Ylangene |
| 75 | Zingiberene |
| 76 | Abieta-8,12-diene |
| 77 | α-Amorphene |
| 78 | alloaromadendrene |
| 79 | Benzyl benzoate |
| 80 | Beyerene |
| 81 | Bisabolene |
| 82 | Isopentyl-2-methylbutanoate |
| 83 | cis-Calamenene |
| 84 | α-Cadinene |
| 85 | τ-Cadinol |
| 86 | 2-Carene |
| 87 | Campholenealdehyde |
| 88 | Caryophyllene oxide |
| 89 | cis-Carveol |
| 90 | ( + ) trans-Carveol |
| 91 | Carvone |
| 92 | α-Cedrene |
| 93 | Cedrol |
| 94 | Cembra-1,3,7,11-tetraene |
| 95 | Cembra-3,7,11,15-tetraene |
| 96 | Cembrene |
| 97 | Cembrene C |
| 98 | Citronellyl acetate |
| 99 | α-Cubebene |
| 100 | β-Cubebene |
| 101 | o-Cymene |
| 102 | Chrysanthenone |
| 103 | 1,4-Cyclohexadiene |
| 104 | p-Cymen-8-ol |
| 105 | Decanol |
| 106 | Decyl acetate |
| 107 | 2,6-Dimethoxytoluene |
| 108 | 3,5-Dimethoxytoluene |
| 109 | Duva-3,9,13-trien-1,5α-diol |
| 110 | Duva-4,8,13-trien-1a,3α-diol |
| 111 | Duva-3,9,13-trien-1,5α-diol-1-acetate |
| 112 | Duva-3,9,13-triene-1α-ol-5,8-oxide-1-acetate |
| 113 | β-Elemene |
| 114 | Farnesyl acetate |
| 115 | Geranyl acetate |
| 116 | α-Gurjunene |
| 117 | Hedycariol |
| 118 | 1,3,6-Trimethylencycloheptane |
| 119 | 1-Hexanol |
| 120 | Hexyl acetate |
| 121 | Hexyl hexanoate |
| 122 | α-Humulene |
| 123 | Incensole |
| 124 | Incensole acetate |
| 125 | Isodurene |
| 126 | Isocembrene |
| 127 | Isophyllocladene (kaur-15-ene) |
| 128 | Kaurene |
| 129 | Ledol |
| 130 | Maaliane |
| 131 | p-Mentha-1,5-dien-8-ol |
| 132 | o-Methyl anisole |
| 133 | α-Muurolene |
| 134 | α-Muurolol |
| 135 | Myrtenal |
| 136 | Naphthalene |
| 137 | Naphthalene 1,2,3,4,4a,7-hexahydro-1,6-dimethyl-4- |
| | (1-methylethyl |
| 138 | Neryl acetate |
| 139 | cis-Nerolidol |
| 140 | (S)-trans-Nerolidol |
| 141 | (E)-Nerolidol |
| 142 | 1-Octanol |
| 143 | n-Octanol |
| 144 | Octanol acetate |
| 145 | Octyl acetate |
| 146 | Octyl formate |
| 147 | allo-Ocimene |
| 148 | Phenanthrene-7-ethenyl-9,10,10a-dodeca-hydro-1-1-4a- |
| | 7-tetramethyl |
| 149 | α-Phellandrene epoxide |
| 150 | Phyllocladene |
| 151 | α-Pinene-epoxide |
| 152 | 1-β-Pinene |
| 153 | 2-β-Pinene |
| 154 | Isopinocampheol |
| 155 | Piperitone |
| 156 | Pyrimidine |
| 157 | Sabinyl acetate |
| 158 | Sandaracopimara-8(14)-15-diene |
| 159 | Sclarene |
| 160 | α-Selinene |
| 161 | β-Selinene |
| 162 | δ-Selinene |
| 163 | trans-Terpine |
| 164 | 4-Terpineol |
| 165 | Terpinolene |
| 166 | Isoterpinolene |
| 167 | 2,4(10)-Thujadiene |
| 168 | Thujopsene |
| 169 | Thunbergol |
| 170 | Isomyl-valerate |
| 171 | Verticilla-4(20),7,11-triene |
| 172 | Verbenone |
| 173 | cis-Verbenol |
| 174 | Verticiol |
| 175 | Viridiflorol |
| 176 | Benzene, 1methoxy-2-methyl |
| 177 | endo-Borneol |
| 178 | γ-Campholene aldehyde |
| 179 | α-Campholene aldehyde |
| 180 | Cara-2,4-diene |
| 181 | Carvacrol |
| 182 | Carvotanacetone |
| 183 | trans-Dihydrocarvone |
| 184 | Cumin alcohol |
| 185 | m-Cymene-8-ol |
| 186 | p-Cymene-9-ol |
| 187 | p-Cymenene |
| 188 | Dodecanol |
| 189 | Eucalyptol |
| 190 | Eucarvone |
| 191 | Isopropyl benzaldehyde |
| 192 | Isopropyl benzalcohol |
| 193 | cis-1,2-Limonene epoxide |
| 194 | 8,9-Limonene epoxide II |
| 195 | 8,9-Limonene-epoxide I |
| 196 | trans-1,2-Limonene epoxide |
| 197 | cis-Linalool oxide |
| 198 | trans-Linalool oxide |
| 199 | p-Mentha-1,5-diene-7-ol |
| 200 | p-Mentha-1,8-diene-4-ol |
| 201 | cis-p-Menth-2-en-1-ol |
| 202 | cis-p-Mentha-1(7),8-diene-2-ol |
| 203 | cis-p-Mentha-2,8-diene-1-ol |
| 204 | trans-p-Menth-2-en-1-ol |
| 205 | trans-p-Mentha-1(7),8-diene-2-ol |
| 206 | trans-p-Mentha-2,8-diene-1-ol |
| 207 | 2,4(8)-p-Menthadiene |
| 208 | p-Mentha-6,8-dien-2-one |
| 209 | p-Methylanisole |
| 210 | Myrtenol |
| 211 | Nerol |
| 212 | trans-Ocimene |
| 213 | (E)-β-Ocimene epoxide |
| 214 | α-Phellandrene-dimer |
| 215 | α-Phellandrene-8-ol |
| 216 | α-Pinene oxide |
| 217 | Pinocamphone |
| 218 | Pinocarvone |
| 219 | Piperitenone |
| 220 | Isopiperitenone |
| 221 | trans-Piperitol |
| 222 | α-Terpineol |
| 223 | Sabina ketone |
| 224 | cis-Sabinene hydrate |
| 225 | trans-Sabinene hydrate |
| 226 | trans-Sabinol |
| 227 | 2,5-Dimethylstyrene |
| 228 | cis-1,2-Epoxy-terpin-4-ol |
| 229 | Thuj-3-en-10-al |
| 230 | Thujanol |
| 231 | Thunbergene |
| 232 | Thymol |
| 233 | Umbellulone |
| 234 | Verticellol |
| 235 | 5,5-Dimethyl-1-vinylbicyclo-[2.1.1]-hexane |
| 236 | p-Anisaldehyde |
| 237 | Aromadendrene |
| 238 | Benzyl tigilate |
| 239 | p-Camphorene |
| 240 | Isocaryophyllene |
| 241 | Cumaldehyde |
| 242 | Cyclosativene |
| 243 | γ-Eudesmol |
| 244 | Guaioxide |
| 245 | 5-Guaiene-11-ol |
| 246 | Isogermacrene D |
| 247 | 4-Methylene-1-(1-methylethyl)-bicyclo[3.1.0]hex-2-ene |
| 248 | 2-Methyl-5-(1-methylethyl)-1,3-cyclohexadiene |
| | monoepoxide |
| 249 | n-Pentadecan |
| 250 | Perilla alcohol |
| 251 | Perillol |
| 252 | Thujol |
| 253 | m-Thymol |
| 254 | α-Ylangene |
| 255 | γ-Campholene aldehyde |
| 256 | n-Decanoic acid |
| 257 | β-Eudesmene |
| 258 | β-Cyclogeranylacetate |
| 259 | n-Hexanoic acid |
| 260 | Hexylcaprylate |
| 261 | Incensyl acetate |
| 262 | Incensole oxide |
| 263 | Incensole oxide acetate |
| 264 | Lauric acid |
| 265 | p-Methylacetophenone |
| 266 | p-Methyleugenol |
| 267 | β-Myrcene |
| 268 | n-Nonanoic acid |
| 269 | n-Octanoic acid |
| 270 | 3,4-Dimethoxystyrene |
| 271 | α-Cadinol |
| 272 | 1,Hydroxy-1,7-dimethyl-4-isopropyl-2,7-cyclodecadiene |
| 273 | 1,5,5,8-Tetramethyl-12-oxabicyclo-[9.1.0]-dodeca-3,7-diene |
| 274 | 1-Methyl-4-(1-methylethenyl)-1,2-cyclohexanediol |
| 275 | trans-p-Mentha-2,8-dienol |
| 276 | 1,2,3,4,6,8a-hexahydro-1-isopropyl-4,7-dimethyl- |
| | naphthalene |
| 277 | 2-Isopropenyl-4a,8-dimethyl-1,2,3,4,4a,5,6,8a- |
| | ctahydronaphthalene |
| 278 | 3,5-Dimethoxytoluene |
| 279 | (Z)-α-Hydroxymanool |
| 280 | Hydroxy-manool |
| 281 | Methyl linoleate |
| 282 | 1-Acetyl-4-isopropenylcyclopentene |
| 283 | 2,4-Dimethylacetophenone |
| 284 | α-Amyrenone |
| 285 | β-Amyrenone |
| 286 | 10-Hydroxy-4-cadinen-3-one |
| 287 | 2-Hydroxy-1,4-cineole |
| 288 | Cryptone |
| 289 | Eucarvone |
| 290 | Isopropylidencyclohexane |
| 291 | 1,2,4-Trihydroxy-p-menthane |
| 292 | Δ⁴-p-Menthen-2-one |
| 293 | 5-Hydroxy-p-menth-6-en-2-one |
| 294 | Myrtenoic acid |
| 295 | Nopinone |
| 296 | 3,6,6,-Trimethyl-norpinan-2-one |
| 297 | o-Methylacetophenone |
| 298 | Perillaaldehyde |
| 299 | Phellandra |
| 300 | Pinocamphone/isopinocamphone |
| 301 | Thujone |
| 302 | 24-Noroleana-3,12-diene |
| 303 | 24-Noroleana-3,9(11),12-triene |
| 304 | 24-Norursa-3,12-diene |
| 305 | 24-Norursa-3,9(11),12-triene |
| 306 | 24-Norursa-3.12-dien-11-one |
| 307 | α-Amyrine |
| 308 | epi-α-Amyrine |
| 309 | β-Amyrine |
| 310 | Lupeol |
| 311 | Terpinenyl acetate |
| 312 | 1,5-Isopropyl-2-methylbicyclo[3.1.0]hex-3-en-2-ol |
| 313 | α-Campholenal |
| 314 | (3E,5E)-2,6-Dimethyl-1,3,5,7-octatetraene |
| 315 | (E)-2,3-Epoxycarene |
| 316 | 3,4-Dimethylstyrene |
| 317 | 1-(2,4-Dimethylphenyl)ethanol |
| 318 | 4-Methylbenzoic acid |
| 319 | p-Menth-1(7)-en-2-one |
| 320 | Caryophyllene |
| 321 | Methylcycloundecanecarboxylate |
| 322 | Nonanoic acid |
| 323 | Hexadecanoic acid |
| 324 | 1,4-Cineol |
| 325 | Sabinene hydrate |
| 326 | Methyl-trans-2-cis-4-decadienoate |
| 327 | 2-Hydroxy-5-methoxy-acetophenone |
| 328 | (E)-β-Farnesene |
| 329 | 2-Dodecenoic acid methyl ester |
| 330 | Calacorene |
| 331 | n-Dodecanoic acid |
| 332 | α-Guaiol |
| 333 | Caryophylla-3(15),7(14)-dien-6-ol |
| 334 | Cadalene |
| 335 | Eudesma-4(15),7-dien-1β-ol |
| 336 | n-Heptadecane |
| 337 | n-Tetradecanoic acid |
| 338 | n-Octadecane |
| 339 | Galaxolide |
| 340 | Manool |

Although many Boswellia species produce Olibanum, the major sources of commercial Olibanum are B. serrata (India), B. sacra (Oman), and B carteri (Somalia). The table below shows the major components of Olibanum derived from diverse Boswellia species, according to their percentage representation:

| Boswellia | | Predominant | Percentage |
|---|---|---|---|
| specie | Source of resin | compound(s) | (%) |
| B. serrata | Commercial (Hamburg, Germany) | Myrcene | 38 |
| B. serrata | NA | α-Thujene | 22.7-47.4 |
| B. serrata | NA | α-Thujene | 29.3 |
| B. serrata | NA | α-Thujene | 61.36 |
| B. carteri | Purchased from the local market of | Duva-3,9,13-triene- | 21.4 |
| | herbs and spices in Egypt | 1a-ol-5,8-oxide-1- | |
| | | acetate | |
| B. sacra | Botanically certified oleogum resin | E-β-Ocimene | 32.3 |
| B. carteri/sacra | NM | Octanol acetate | 45.2 |
| B. carteri | Authentic sample from Ethiopia | Octyl acetate | 39.3 |
| | certified for its authenticity from the | | |
| | Agricultural Department of the | | |
| | Ethiopian government | | |
| B. rivae | NA | Limonene | 28.0 |
| B. rivae | Authentic sample from Ethiopia | α-Pinene | 16.7 |
| B. rivae | NA | α-Pinene | 13.3 |
| B. rivae | NA | Octanol | 17.8 |
| B. neglecta | NA | α-Pinene | 16.7 |
| B. neglecta | Authentic sample from Ethiopia | α-Pinene | 21.3 |
| B. papyrifera | NA | Octyl acetate | 63.5 |
| B. papyrifera | NA | Octyl acetate | 56.0 |
| B. pirottae | NA | Trans-Verbenol | 15.5 |
| B. pirottae | NA | Terpinen-4-ol | 14.6 |
| B. frereana | Commercial (Hamburg, Germany) | α-Pinene | 38.0 |

One exemplary analysis of Olibanum has indicated the following components
Acid resin (6%), soluble in alcohol and having the formula C₂₀H₃₂O₄
gum (similar to gum arabic) 30-36%
3-acetyl-beta-boswellic acid (Boswellia sacra)
alpha-boswellic acid (Boswellia sacra)
incensole acetate, C₂₁H₃₄O₃
phellandrene

Another analysis of B. serrata resin revealed that the resinous part of Boswellia serrata contains monoterpenes (α-thujene); diterpenes (macrocyclic diterpenoids such as incensole, incensole oxide, iso-incensole oxide, a diterpene alcohol [serratol]); triterpenes (such as α- and β-amyrins); pentacyclic triterpenic acids (boswellic acids); tetracyclic triterpenic acids (tirucall-8,24-dien-21-oic acids). The structures of four major pentacyclic triterpenic acids (boswellic acids) as also some of their characteristic features of four pentacyclic triterpene acids (Boswellic acid) are given in the following table:

| | | Acetyl- | 11-keto- | Acetyl-11-keto- |
|---|---|---|---|---|
| Properties | B-Boswellic acid | B-Boswellic acid | B-Boswellic acid | B-Boswellic acid |
| Molecular formula | C [symbol] H [symbol] O [symbol] | C [symbol] H [symbol] O [symbol] | C [symbol] H [symbol] O [symbol] | C [symbol] H [symbol] O [symbol] |
| Molecular weight | 456.7 | 498.74 | 470.09 | 512.73 |
| Chemical name | 3α-Hydroxy-urs- | 3α-Acetoxy-urs- | 3α-Hydroxy-urs-12- | 3α-Acetoxy-urs-12- |
| | 12-en-23-olc acid | 12-en-23-olc acid | en-11-keto-23-olc acid | en-11-keto-23-olc acid |
| Melting point | 226-228° | 252-255° | 195-197° | 271-274° |
| Specific roatation | +106.8° | +138° | +78.5° | +88.5° |
| UV-MeOH | Maxima at 208 nm | Maxima at 208 nm | Maxima at 250 nm | Maxima at 250 nm |
| NMR | 5.15, CH═C, 4.08, | 5.31, CH═C, 5.2, | 5.55, CH═C, 4.08, | 5.55, CH═C, 5.2, |
| (In CDCl₃, δ ppm) | CH—OH; | CH—OAc; | CH—OH; | CH—OAc; |
| | 2.3-1.1, Methylenes and | 2.1, COCH₃, 1.9-1.25, | 2.6-1.4, Methylenes and | 2.6-1.4, Methylenes and |
| | methines, 23 protons; | Methylenes and methines, | methines, 21 protons; | methines, 21 protons; |
| | 1.1-0.7 Methyls, | 23 protons, 1.2-0.7, | 1.25-0.75, Methyls 21 | 1.25-0.75 Methyls, |
| | 21 protons | Methyls, 21 protons | protons | 21 protons |
| FTIR | 3500 (OH), | 1732 (OAc), | 3460 (OH), | 1740 (Ac), 1701 (COOH), |
| (In KBr, cm⁻¹) | 1699.5 (COOH) | 1701 (COOH) | 1693 (COOH) | 647(α,β-unsaturated |
| | | | | carbonyl) |
| GC MS | 394 (M-68[44 due to | 394 (M-104[44 due | 408 (M-68[44 due | 408 (M-68[44 due |
| | —CO₂ and 18 | to —CO₂ and 60 | to —CO₂ and | to —CO₂ and 18 |
| | due to —H₂O]); | due to HOAc]); | 18 due to —H₂O]); | due to HOAc]); |
| | Other fragments; | 218 (base peak) | 232 (base peak); | 232 (base peak); Other |
| | 203, 189, 175, 161 | | Other fragments; | fragments: 217, 175, |
| | | | 217, 175, 161, 135 | 161, 135 |

[symbol]

indicates data missing or illegible when filed

The Olibanum gum component contains polysaccharides and polymeric components. The proteoglycans in Olibanum comprise mainly D-galactose units in the main chain and glucuronic acid, uronic acids, 4-O-methyl-glucuronic acid and arabinose in the side chains.

According to a specific embodiment, the active ingredient or combination thereof includes an alcohol soluble acid resin, a water soluble gum, an alpha-boswellic acid, an incensole acetate and a phellandrene.

According to a specific embodiment, the active ingredient or combination thereof includes a volatile compound, e.g. α-Thujene, Duva-3,9,13-triene-1a-ol-5,8-oxide-1-acetate, E-β-Ocimene, Octanol acetate, Octyl acetate, Limonene, α-Pinene, Octanol, Trans-Verbenol and Terpinen-4-ol.

According to a specific embodiment, the active ingredient or combination thereof includes a mineral, e.g., potassium, calcium, magnesium, phosphorus, aluminum, iron, sodium, boron, zinc, cadmium, selenium.

According to a specific embodiment, a water or alcohol extract is performed.

In some embodiments, the Olibanum is prepared by water extract. An exemplary water extract is described herein:

Preparation of olibanum extract by water. At first, Olibanum is carefully powdered. The powder (25 g) is mixed with 200 ml of deionized water and stirred with 800 rpm overnight at room temperature. This mixture is centrifuged at 1,500 rpm for 10 min and the supernatant collected. Thereafter, the supernatant is again centrifuged at 2,500 rpm for 10 min and successively at 10,000 rpm for 20 min, and then filtered. The filtrates can be stored at −20 C and then freeze-dried −58 C and 0.5 Torr for 24 h to yield 4.02 gr of water soluble extract. At the next step, the resulted powder is dissolved in 100 ml methanol and stirred for 12 hr. at room temperature, then allowed to settle. The precipitate phase is collected and dried in an oven. Again the powder is dissolved in deionized water, centrifuged repeatedly and refiltered. The filtrates can be stored and then freeze-dried.

In some embodiments, the Olibanum is prepared by alcohol extract. An exemplary alcohol extract is described herein:

Preparation of olibanum extract by alcohol: In this method, 100 gr of Olibanum powder with 400 ml of methanol is mixed. This mixture is then stirred at 650 rpm for 24 hours. The resulting mixture is made up of two phases, the upper phase is alcoholic and yellow, and contains substances that are soluble in alcohol. The material is then dried in an oven at 50 C. The bottom phase has a sedimentary and white state, which is set to in the oven until dry. The resulting powder in the water is well dissolved and the obtained solution is centrifuged at 1,500 rpm for 10 min and the supernatant collected. Thereafter, the supernatant is again centrifuged at 2,500 rpm for 10 min and successively at 10,000 rpm for 20 min, and then filtered. The filtrates can be stored at −20 C and then freeze-dried.

Other extraction procedures include, but are not limited to, those described in Mertens et al, et al. 2009, Flavor and Fragrance, 24:279-300 and Hamm et al, Phytochemistry 2005, 66:1499-1514, which are hereby incorporated by reference in their entirety.

Also contemplated herein are Olibarum and other compositions from trees of the genus Boswellia.

Examples include, but are not limited to:

Some Boswellia
Species
B. socotrana
B. elongata
B. ameero
B. carteri
B. neglecta
B. sacra
B. thurifera
B. frereana
B. dioscorides
B. rivae
B. papyrifera
B. serrata

Chemical Composition of Gynostemma pentaphyllum (Jiaogulan)

Gynostemma pentaphyllum is a perennial herb from the Cucurbitaceae family, with 5-lobed leaves and a gourd-like, inedible fruit which grows in forests, thickets or roadsise on mountain slopes in many areas of Northeast and Southeast Asia, including China, Taiwan, S Korea, Japan, Thailand, Vietnam and Laos. G. pentphyllum also grows in Bangladesh, Bhutan, India, Indonesia, Malaysia, Myanmar, Nepal, New Guinea and Sri Lanka. Jiaogulan is prized for its reputation as a “longevity plant”. Characterization and identification of chemical compounds of Gynostemma pentaphyllum using a variety of methods identified a large variety of compounds in Gynostemma pentaphyllum (Thun.) Makino and classified them as generally being:
Saponin Glycosides (e.g., gypenosides)
Phenolic compounds
Flavenoids (e.g. Kaempferol, quercetin, rutin, ombuin, isorahmnetin)
Polysaccharides
Sterols (e.g. ergostane, cholestane, stigmastane)
Trace elements (e.g. Cu, Fe, Zn, Mn, Co, Ni, Se, Mo and Sr)
Carotenoids
Volatiles (e.g. malonic acid, benzyl-O-beta-D-glucopyranoside, lutein, vomifoliol, palmitic acid, linoleic acid)

According to specific embodiments, the saponin compounds in Jiaogulan and the polysaccharide compounds are the compounds that constitute its phytochemical activity. The most abundant saponin compound in Jiaogulan was found to be gypenoside.

Most Jiaogulan saponins belong to a family of triterpenoid saponins. They are also referred to as gypenosides, and dammarane derivatives. More than 150 saponins have been isolated from G. pentaphyllum plants. Saponins have been identified in Jiaogulan leaves and stems, flower buds, fruits, berries, and seeds.

The table below shows the phytochemical properties of 5 different Gynostemma pentaphyllum samples from different sources:

| | | TPC | TSC | TFC | RUTIN | QUERCITIN | R + Q |
|---|---|---|---|---|---|---|---|
| | | (mg | (mg | (mg | CONTENT | CONTENT | (umol |
| SAMPLE | SOLVENT | GAE/g) | GE/g) | RE/g) | (ug/g) | (ug/g) | QE/g) |
| GP1 | 50% acetone | 44.3 | 38.02 | 21.44 | 3049.5 | 4906.5 | 21.2 |
| | 50% ethanol | 37.5 | 41.39 | 26.40 | 7948.2 | 7431.8 | 37.6 |
| | 100% ethanol | 33.6 | 87.28 | 26.87 | 11235.4 | 7279.1 | 42.5 |
| GP2 | 50% acetone | 14.9 | 90.17 | 10.6 | 2527.3 | 117.5 | 4.5 |
| | 50% ethanol | 12.9 | 114.48 | 14.27 | 3588.1 | 136.2 | 6.3 |
| | 100% ethanol | 6.9 | 132.57 | 13.84 | 2131.9 | 166.2 | 4.0 |
| GP3 | 50% acetone | 12.3 | 47.62 | 10.52 | 8614.9 | 358.9 | 15.3 |
| | 50% ethanol | 10.6 | 59.13 | 9.51 | 9954.0 | 411.0 | 17.7 |
| | 100% ethanol | 6.7 | 64.57 | 8.05 | 7193.0 | 549.4 | 13.6 |
| GP4 | 50% acetone | 43.2 | 77.64 | 63.48 | 1409.2 | 241.3 | 3.1 |
| | 50% ethanol | 30.4 | 82.12 | 54.04 | 680.2 | 150.8 | 1.6 |
| | 100% ethanol | 17.7 | 104.1 | 36.47 | 579.4 | 151.3 | 1.4 |
| GP5 | 50% acetone | 13.1 | 23.61 | 14.55 | nd | nd | |
| | 50% ethanol | 10.2 | 60.7 | 16.53 | nd | nd | |
| | 100% ethanol | 8.9 | 123.97 | 22.11 | nd | nd | |

GP1-5 represent G. pentaphyllum samples from different sources. Data are per gram of dry botanical basis and are expressed as mean (SD. Different letters represent significant differences (P < 0.05). nd stands for not detectable. TPC, TSC, and TFC stand for total phenolic content, total saponin content, and total flavonoid content by spectrometric methods, respectively. GAE, GE, RE, and QE stand for gallic acid equivalents, gypenoside equivalents, rutin equivalents, and quercetin equivalents. Rutin and quercetin contents were flavonoid profile obtained by HPLC. R + Q stands for total amount of rutin and quercetin.
Ethanol extraction: 12 g sample in 250 ml 100% ethanol, 5 hours in Soxhlet apparatus.
50% acetone extraction and 75% ethanol extraction: 2 g sample in 20 ml solvent at ambient temperature and filtration through 45 micron filter.

Water content of the Jiaogulan samples ranged from 3.79 to 7.57 g/100 g sample. Dietary fiber content ranged from 0.6 g/g to 0.24 g/g sample. Selenium content ranged from 1.7 mg/kg to 0.94 mg/kg.

According to a specific embodiment, the active ingredient or combination thereof includes a gypenoside. Some specific ginsenosides include, but are not limited to CP-1-6.

According to a specific embodiment, the active ingredient or combination thereof includes a volatile compound, e.g., malonic acid, benzyl-O-beta-D-glucopyranoside, lutein, vomifoliol, palmitic acid, linoleic acid.

According to a specific embodiment, the active ingredient or combination thereof includes a phytosterol, e.g., stigmasterol, ergostane.

According to a specific embodiment, the active ingredient or combination thereof includes a flavenoid, e.g., Kaempferol, quercetin, rutin.

According to a specific embodiment, the active ingredient or combination thereof includes a phenolic compound.

According to a specific embodiment, the active ingredient or combination thereof includes a mineral, e.g., potassium, calcium, magnesium, phosphorus, aluminum, iron, sodium, boron, zinc, cadmium, selenium.

According to a specific embodiment, the active ingredient or combination thereof includes a vitamin, e.g., vitamin D, vitamin A and vitamin C.

According to a specific embodiment, a methanol or ethanol extract is performed, e.g., ethanol concentration is 100 or 75%; 5 hours in Soxhlet apparatus, or 50% acetone extraction and 75% ethanol extraction: 2 g sample in 20 ml solvent at ambient temperature and filtration through 45 micron filter. Other extraction procedures include, but are not limited to, those described in Yantao et al. 2016 Chi Med 11:43, which is hereby incorporated by reference in its entirety.

According to another embodiment, the plant part is leaf.

Also contemplated herein are plants of the genus Gynostemma.

Origanum syriacum

According to a specific embodiment, the plants of this species include flavones, monoterpenoids and monoterpenes. Over 60 different compounds have been identified, with the primary ones being carvacrol and thymol ranging to over 80%, while lesser abundant compounds include p-cymene, γ-terpinene, caryophyllene, spathulenol, germacrene-D, β-fenchyl alcohol and δ-terpineol.

The table below shows a profile of the organic compounds identified in Origanum extract through fractional distillation:

Profile of the organic compounds found in the fractions analyzed.

| | | | % de Relative Area | | | | | |
|---|---|---|---|---|---|---|---|---|
| Compound | Boiling Point ° C. | Code | F1 | F2 | F3 | F4 | Unoil | Ooil |
| α-thujene | 150-152 | MH1 | 5.03 | 0.389 | ND | ND | | 1.74 |
| α-pinene | 156 | MH2 | 3.01 | ND | ND | ND | ND | 1.07 |
| β-myrcene | 166-168 | MH3 | 11.62 | 6.93 | 1.08 | ND | ND | 5.50 |
| Phellandrene | 172 | MH4 | 1.32 | 1.00 | ND | ND | ND | 0.72 |
| α-terpinene | 174 | MH5 | 8.91 | 8.32 | 2.90 | ND | ND | 5.57 |
| o-cymene | 174 | MH6 | 47.96 | 53.97 | 38.14 | 1.31 | 0.973 | 39.13 |
| Limonene | 175 | MH7 | 2.29 | 2.71 | 1.25 | ND | ND | 1.58 |
| 1,8-cineole | 177 | MO1 | 1.51 | 1.77 | 2.74 | ND | ND | 1.53 |
| γ-terpinene | 181-183 | MH8 | 15.59 | 24.43 | 40.57 | 1.40 | 0.94 | 22.34 |
| Thymol | 232 | MO2 | ND | ND | ND | 5.08 | 3.77 | 1.71 |
| Carvacrol | 237-238 | MO3 | ND | ND | 4.58 | 60.03 | 64.31 | 12.60 |
| Trans-caryophyllene | 268 | SeH1 | ND | ND | 2.97 | 18.96 | 13.78 | 3.47 |
| α-humulene | 276 | SeH2 | ND | ND | 0.34 | 6.16 | 8.36 | 1.56 |
| Monoterpene hydrocarbons | | (MH) | 95.73 | 97.75 | 83.94 | 2.71 | 1.91 | 77.65 |
| Monoterpene oxygenated | | (MO) | 1.51 | 1.77 | 7.32 | 65.11 | 68.08 | 15.84 |
| Sesquiterpene hydrocarbons | | (SeH) | ND | ND | 3.31 | 25.12 | 22.14 | 5.03 |
| Total identified components | | | 97.24 | 99.52 | 94.57 | 92.94 | 92.13 | 98.52 |

Oregano essential oil (Ooil) was obtained through the steam entrainment method and the oil fractions through a fractional distillation system. The first fraction started to distill at a temperature of 82° C. and the last fraction distilling at 140° C., finally undistilled oil (Unoil) was obtained. At the end of the process, five fractions named Fraction 1 (F1), Fraction 2 (F2), Fraction 3 (F3), Fraction 4 (F4), and undistilled oil (Unoil) were obtained.

When Origanum extract was analyzed on HPLC, a variety of phenolic compounds were identified:

Phenolic compounds determined by the HPLC method in O. vulgare ssp. vulgare extract.

| | [M—H]⁻, | Retention Time | UV | MS | Concentration |
|---|---|---|---|---|---|
| Compounds | m/z | (t_{R}), min | Detection | Detection | (mg/g) |
| Gentisic acid | 153 | 3.69 ± 0.04 | NO | YES | <0.02 |
| Chlorogenic acid | 353 | 6.43 ± 0.05 | YES | YES | 2.10 ± 0.14 |
| p-Coumaric acid | 163 | 9.48 ± 0.08 | NO | YES | <0.02 |
| Hyperoside | 463 | 18.60 ± 0.12 | YES | YES | 1.05 ± 0.03 |
| Isoquercitrin | 463 | 20.29 ± 0.10 | YES | YES | 0.71 ± 0.19 |
| Rutin | 609 | 20.76 ± 0.15 | YES | YES | 0.64 ± 0.15 |
| Rosmarinic acid | 360 | 21.80 ± 0.10 | YES | YES | 12.83 ± 2.19 |
| Quercitrin | 447 | 23.64 ± 0.13 | YES | YES | 0.50 ± 0.08 |
| Quercetin | 301 | 27.55 ± 0.15 | NO | YES | <0.02 |
| Luteolin | 285 | 29.64 ± 0.19 | YES | YES | 0.10 ± 0.04 |

Values are the mean ± SD (n = 3).

Total polyphenol content and antioxidant activity of O. vulgare ssp. vulgare extract.

| | TPC (mg | Flavonoid | Caffeic Acids | CUPRAC | FRAP | SO Scavenging |
|---|---|---|---|---|---|---|
| Sample | GAE/g) | (mg RE/g) | (mg CAE/g) | (μM TE/g) | (μM TE/g) | (μM TE/g) |
| O. vulgare | 94.69 ± 4.03 | 38.46 ± 3.54 | 29.92 ± 1.08 | 1284 ± 66 | 794.40 ± 25.80 | 44.00 ± 0.56 |

Each value is the mean ± SD of three independent measurements. TPC, total polyphenols content; SO, superoxide; GAE, gallic acid equivalents; RE, rutin equivalents; CAE, caffeic acid equivalents; TE, Trolox equivalents.

Also contemplated herein are plants of the genus Origanum.

Origanum is a genus of herbaceous perennials and subshrubs in the family Lamiaceae, native to Europe, North Africa, and much of temperate Asia, where they are found in open or mountainous habitats. A few species also naturalized in scattered locations in North America and other regions.

The plants have strongly aromatic leaves and abundant tubular flowers with long-lasting coloured bracts. The genus includes the important group of culinary herbs: marjoram (Origanum majorana) and oregano (Origanum vulgare).

Examples include, but are not limited to:
Origanum acutidens (Hand.-Mazz.) Ietsw.—Turkey, Iraq
Origanum x adanense Baser & H. Duman—Turkey (O. bargyli x O. laevigatum)
Origanum x adonidis Mouterde—Lebanon (O. libanoticum x O. syriacum subsp. bevanii)
Origanum akhdarense Ietsw. & Boulos—Cyrenaica region of eastern Libya
Origanum amanum Post—Hatay region of Turkey
Origanum x barbarae Bornm.—Lebanon (O. ehrenbergii x O. syriacum subsp. bevanii)
Origanum bargyli Mouterde—Turkey, Syria
Origanum bilgeri P. H. Davis—Antalya region of Turkey
Origanum boissieri Ietsw.—Turkey
Origanum calcaratum Juss.—Greece
Origanum compactum Benth.—Spain, Morocco
Origanum cordifolium (Montbret & Aucher ex Benth.) Vogel—Cyprus
Origanum cyrenaicum Bég. & Vacc.—Cyrenaica region of eastern Libya
Origanum dayi Post—Israel
Origanum dictamnus L.—hop marjoram, Cretan dittany, dittany of Crete—endemic to Crete
Origanum x dolichosiphon P. H. Davis—Seyhan region of Turkey (O. amanum x O. laevigatum)
Origanum ehrenbergii Boiss.—Lebanon
Origanum elongatum (Bonnet) Emb. & Maire—Morocco
Origanum floribundum Munby—Algeria
Origanum x haradjanii Rech. f—Turkey (O. laevigatum x O. syriacum subsp. bevanii)
Origanum haussknechtii Boiss.—Turkey
Origanum husnucan-baseri H. Duman, Aytac & A. Duran—Turkey
Origanum hypericifolium O. Schwarz & P. H. Davis—Turkey
Origanum x intercedens Rech. f.—Greece, Turkey (O. onites x O. vulgare subsp. hirtum)
Origanum x intermedium P. H. Davis—Denizli region of Turkey (O. onites x O. sipyleum)
Origanum isthmicum Danin—Sinai
Origanum jordanicum Danin & Kunne—Jordan
Origanum laevigatum Boiss.—Turkey, Syria, Cyprus
Origanum leptocladum Boiss.—Turkey
Origanum libanoticum Boiss.—Lebanon
Origanum majorana L.—(sweet) marjoram—Turkey, Cyprus; naturalized in scattered locations in Europe, North Africa, North+South America
Origanum x lirium Heldr. ex Halácsy—Greece (O. scabrum x O. vulgare subsp. hirtum)
Origanum x majoricum Cambess.—hardy sweet marjoram—Spain including Balearic Islands (O. majorana x O. vulgare subsp. virens)
Origanum microphyllum (Benth.) Vogel—Crete
Origanum x minoanum P. H. Davis—Crete (O. microphyllum x O. vulgare subsp. hirtum)
Origanum minutiflorum O. Schwarz & P. H. Davis—Turkey
Origanum munzurense Kit Tan & Sorger—Turkey
Origanum x nebrodense Tineo ex Lojac—Sicily (O. majorana x O. vulgare subsp. viridulum)
Origanum onites L.—Greece, Turkey, Sicily
Origanum x pabotii Mouterde—Syria (O. bargyli x O. syriacum subsp. bevanii)
Origanum pampaninii (Brullo & Furnari) Ietsw—Cyrenaica region of eastern Libya
Origanum petraeum Danin—Jordan
Origanum punonense Danin—Jordan
Origanum ramonense Danin—Israel
Origanum rotundifolium Boiss.—Turkey, Caucasus
Origanum saccatum P. H. Davis—Turkey
Origanum scabrum Boiss. & Heldr. in P. E. Boissier—Greece
Origanum sipyleum L.—Turkey, Greek Islands
Origanum solymicum P. H. Davis—Antalya region of Turkey
Origanum symes Carlström—Islands of the Aegean Sea
Origanum syriacum L.—Turkey, Cyprus, Syria, Lebanon, Jordan, Palestine, Israel, Sinai, Saudi Arabia
Origanum vetteri Briq. & Barbey—Crete
Origanum vogelii Greuter & Burdet—Turkey
Origanum vulgare L.—oregano—Europe, North Africa, temperate Asia (Iran, Siberia, Central Asia, China, etc.); naturalized in parts of North America, New Zealand, Venezuela.

According to a specific embodiment, the active ingredient or combination thereof includes an organic compound component of Origanum extract.

According to a specific embodiment, the active ingredient or combination thereof is selected from the group consisting of α-thujene α-pinene, β-myrcene, Phellandrene, α-terpinene, o-cymene, Limonene, 1,8-cineole, γ-terpinene, Thymol, Carvacrol, Trans-caryophyllene and α-humulene.

According to a specific embodiment, the active ingredient or combination thereof includes a monoterpene hydrocarbon, an oxygenated monoterpene and a sesquiterpene hydrocarbon.

According to a specific embodiment, the active ingredient or combination thereof includes a phenolic compound, e.g., gentisic acid, chlorogenic acid, p-coumaric acid, hyperoside, isoquercitrin, rutin, rosmarinic acid, quercirtin, quercetin and luteolin.

According to a specific embodiment, the active ingredient or combination thereof includes a mineral, e.g., potassium, calcium, magnesium, phosphorus, aluminum, iron, sodium, boron, zinc, cadmium, selenium.

Sesame

Sesame seeds contain thelignans, sesamolin, sesamin, pinoresinol andlariciresinol. Insoluble 11S globulin and soluble 2S albumin, conventionally termed α-globulin and β-globulin, are the two major storage proteins and constitute 80-90% of total seed proteins in sesame. Comparison of amino acid composition indicated that they are substantially less hydrophobic than the known oleosins, and thus should not be aggregated multimers of oleosins. The results of immuno-recognition to sesame proteins reveals that these three polypeptides are unique proteins gathered in oil bodies, accompanying oleosins and triacylglycerols, during the active assembly of the organelles in maturing seeds. The phospholipid, oleic and linoleic acids, chlorophyll and sesamolin, sesamol and γ-tocopherol are found. 10 compounds [2-furfurylthiol, 2-phenylethylthiol, 2-methoxyphenol, 4-hydroxy2, 5-dimethyl-3[2H]-furanone, 2-pentylpyridine, 2-ethyl-3,5-dimethylpyrazine, acetylpyrazine, [E,E]-2,4-decadienal, 2-acetyl-1-pyrroline and 4-vinyl-2-methoxy-phenol] are quantified. On the basis of high OAVs in oil, especially 2-acetyl-1-pyrroline [roasty], 2-furfurylthiol [coffee-like], 2-phenylethylthiol [rubbery] and 4-hydroxy-2,5-dimethyl3[2H]-furanone [caramel-like] are elucidated as important contributors to the overall roasty, sulphury odour of the crushed sesame material. The structures of novel sesaminol glucosides isolated from sesame seed are determined to be sesaminol 2′-O-β-d-glucopyranoside, sesaminol 2′-O-β-d-glucopyranosyl [1→2]-O-β-dglucopyranoside and sesaminol 2′-O-β-d-glucopyranosyl [1»2]-O-[β-d-glucopyransyl [1»6]]-[β-dglucopyranoside. Also minor sesame lignans such as -(7S,8′R,8R)-acuminatolide piperitol and pinoresinol (as mentioned).

Also contemplated herein are plants of the genus Sesamum.

Examples include, but are not limited to:
Sesamum abbreviatum Merxm.
Sesamum alatum Thonn.
Sesamum angolense Welw.
Sesamum biapiculatum De Wild.
Sesamum calycinum Welw.
Sesamum capense Burm. f.
Sesamum digitaloides Welw. ex Schinz
Sesamum gracile Endl.
Sesamum hopkinsii Suess.
Sesamum indicum L.
Sesamum lamiifolium Engl.
Sesamum latifolium J. B. Gillett
Sesamum lepidotum Schinz
Sesamum macranthum Oliv.
Sesamum marlothii Engl.
Sesamum mombazense De Wild. & T. Durand
Sesamum parviflorum Seidenst.
Sesamum pedalioides Welw. ex Hiern
Sesamum radiatum Schumach. & Thonn.
Sesamum rigidum Peyr.
Sesamum rostratum Hochst.
Sesamum sabulosum A. Chev.
Sesamum schinzianum Asch.
Sesamum somalense Chiov.
Sesamum thonneri De Wild. & T. Durand
Sesamum triphyllum Welw. ex Asch.

Plants that contain Lignan according to some embodiments of the invention include a wide variety of plant foods, including seeds (flax, pumpkin, sunflower, poppy, sesame), whole grains (rye, oats, barley), bran (wheat, oat, rye), beans, fruit (particularly berries), and vegetables (Broccoli and curly kale are rich sources of lignans. Other vegetables such as white and red cabbage, Brussels sprouts, cauliflower, carrots, green and red sweet peppers are also good sources).

Additional plants that contain Sesamin include but are limited to Eleutherococcus senticosus.

Thus, any combination of the above plants is contemplated including 2, 3, 4, 5, 6, 7 of the plants. According to another embodiment, a combination of extracts or fractions including 2, 3, 4, 5, 6, 7 of the different plants.

Examples include, but are not limited to, Nigella sativa, Thymus vulgaris, Origanum syriacum, Thymbra spicata, Satujera thymbra, Sesamum indicum and Rhus coriaria.

Nigella sativa, Thymus capitatus, Origanum syriacum, Thymbra spicata, Satujera thymbra, Sesamum indicum and Rhus coriaria.

Nigella sativa, Thymus capitatus, Thymus vulgaris, Thymbra spicata, Satujera thymbra, Sesamum indicum and Rhus coriaria.

Nigella sativa, Thymus capitatus, Thymus vulgaris, Origanum syriacum, Satujera thymbra, Sesamum indicum and Rhus coriaria.

Nigella sativa, Thymus capitatus, Thymus vulgaris, Origanum syriacum, Thymbra spicata, Sesamum indicum and Rhus coriaria.

Nigella sativa, Thymus capitatus, Thymus vulgaris, Origanum syriacum, Thymbra spicata, Satujera thymbra, and Rhus coriaria.

Nigella sativa, Thymus capitatus, Thymus vulgaris, Origanum syriacum, Thymbra spicata, Satujera thymbra, Sesamum indicum.

Nigella sativa, Origanum syriacum, Thymbra spicata, Satujera thymbra, Sesamum indicum and Rhus coriaria.

Nigella sativa, Thymus capitatus, Thymus vulgaris, Satujera thymbra, Sesamum indicum and Rhus coriaria.

Nigella sativa, Thymus capitatus, Thymus vulgaris, Origanum syriacum, Sesamum indicum and Rhus coriaria.

Nigella sativa, Thymus capitatus, Thymus vulgaris, Origanum syriacum, Thymbra spicata, and Rhus coriaria.

Nigella sativa, Thymus capitatus, Thymus vulgaris, Origanum syriacum, Thymbra spicata, Satujera thymbra.

Nigella sativa, Thymus capitatus.

Nigella sativa, Thymus vulgaris.

Nigella sativa, Origanum syriacum.

Nigella sativa, Thymbra spicata.

Nigella sativa, Satujera thymbra.

Nigella sativa, Sesamum indicum.

Nigella sativa, Rhus coriaria.

Also contemplated are various combinations without Nigella sativa.

According to another embodiment, a combination of active ingredients e.g., thymoquinone, carvacrol, thymol; thymoquinone, carvacrol; thymoquinone, thymol; carvacrol, thymol.

Nigella sativa, Thymus capitatus, Thymus vulgaris.

Nigella sativa, Thymus vulgaris, Origanum syriacum.

Nigella sativa, Origanum syriacum, Thymbra spicata.

Nigella sativa, Thymbra spicata, Satujera thymbra.

Nigella sativa, Satujera thymbra, Sesamum indicum Rhus coriaria.

According to some embodiments the plants and active ingredients thereof are listed in the Table below.

| Origanum | Carvacrol |
|---|---|
| Syricaum | thymol |
| Thymus Capitatus | Carvacrol |
| | p-cymene |
| | y-terpinene |
| | b-caryophyllene |
| Thymus Vulgaris | Thymol |
| Thymbra Spicata | Carvacrol |
| | y-terpinene |
| | p-cymene |
| Satureja Thymbra | y-terpinene |
| | p-cymene |
| | carvacrol |
| | thymol |
| Sumac ’ | Tannin |
| Seasame | Lignans |
| | Seasamolin |
| | Seasamin |
| | Pinoresinol |
| | Lariciresinol |
| Nigella sativa | Thymoquinone |

Other embodiments, which comprise any of the Nigella sativa, Thymus capitatus, Thymus vulgaris, Origanum syriacum, Thymbra spicata, Satujera thymbra, Sesamum indicum and Rhus coriaria plants or genera thereof in combinations of 2, 3, 4, 5, 6, 7 and 8 plants are contemplated herein.

Other embodiments of the present invention include in the composition Bromelain or products thereof.

Other embodiments of the present invention include in the composition Pineapple extracts comprising Bromelain.

According to some embodiments of the invention, the component comprises tryptophan or an analog thereof.
According to an aspect of the invention there is provided a food supplement, composition or extracts further including “Beduin Tea” comprising
Rose Leaves Micromeria fruticose, Salvia, cymbopgon (Citral) Aloysia, Verbena officinalis, Origanum majorana, menthe
According to an aspect of the invention there is provided a food supplement, composition or extracts further including “Beduin Tea” comprising
Thyme, sage, cardamom, cinnamon, black tea, habuk, Marmaya.

The plant part, extract thereof, fraction thereof, active ingredient thereof, synthetic analog thereof, mimetic thereof or combination thereof can be used in the treatment of NMRD.

As used herein, “non-malignant respiratory disease” (NMRD) refers to a group of acute and chronic conditions affecting the respiratory tract and interfering with gas exchange, exclusive of cancerous and malignant proliferative pulmonary disease.

Examples of NMRD which are contemplated herein include, but are not limited to, pulmonary hypertension, interstitial lung disease, acute lung injury (ALI), acute respiratory distress syndrome (ARDS), sarcoidosis of the lung, bronchiectasis, asbestosis, berylliosis, silicosis, anthracosis, Histiocytosis X, pneumotitis, smoker's lung, bronchiolitis obliterans, lung scarring due to tuberculosis and pulmonary fibrosis, chronic obstructive pulmonary disease, pneumoconiosis, traumatic pulmonary injury, pulmonary infections and pulmonary manifestations of connective tissue diseases, including systemic lupus erythematosus, rheumatoid arthritis, scleroderma, polymyositis and dermatomyositis.

In some embodiments, the NMRD is the result of a pulmonary infection. Exemplary pulmonary infections which can cause NMRD include bronchitis, pneumonia, bronchiolitis, influenza, tuberculosis and malaria. Pulmonary infections which can cause NMRD include, but are not limited to viral, bacterial, fungal or parasitic infection. Pathogenic agents of pulmonary infections include, but are not limited to influenza viruses A, B and C, Streptococcus pneumonia, Haemophilus influenza, Mycoplasma pneumoniae, Respiratory Syncytial Virus, Pneumocystis jirovecii, Aspergillus, Histoplasma capsulatum, Plasmodium falciparum and parasitic helminth species.

In particular embodiments, types of NMRD contemplated herein include Chronic Obstructive Pulmonary Disease (COPD), Idiopathic Pulmonary Fibrosis (IPF) and Asthma.

In some embodiments, NMRD is the result of a primarily pulmonary disease, as in diseases caused by chronic inhalation of silica, anthracite, toxic fumes, asbestos, etc. In other embodiments, NMRD can be a secondary manifestation of pulmonary injury or dysfunction caused by scarring, fibrosis and other effects of non-pulmonary diseases or conditions, such as connective tissue disease (e.g. RA, scleroderma), infectious disease (e.g. polio) or autoimmune disease (SLA).

Individuals suffering from NMRD typically experience coughing, shortness of breath, wheezing, difficulty in breathing, pulmonary or nasal congestion, copious mucus, inflammation of the airways, chest tightness and exercise intolerance. Moderate to severe cases of NMRD, and particularly in older individuals, those with underlying medical problems and individuals frequently exposed to common triggers of respiratory disease such as particulate pollution (e.g. coal dust, asbestos, silica, etc), dust mites and allergens are more likely to develop into serious, chronic illness, in many cases requiring intensive care with ventilation and life-threatening disease.

According to specific embodiments, the NMRD is COPD.

The plant-derived component or components of the present invention can be co-administered with other medications to increase therapeutic bioavailability, boost therapeutic efficacy, and minimize side effects. The plant-derived component or components of the present invention may be administered in a linear or cyclical form, or in any conformation deemed physiologically appropriate as a means of conveying treatment.

In addition to targeted vasodilation, targeted anti-coagulation can be accomplished. For example, in a disease like acute lung injury, which is often marked by pulmonary intra-alveolar coagulation, targeted anti-coagulation can be delivered to the affected pulmonary area by co-administering an effective dose of the plant-derived component or components of the present invention with an anti-coagulant such as tissue factor pathway inhibitor (TFPI) or site-inactivated factor VIIa in a minimal dose to achieve targeted pulmonary anticoagulation with minimal changes in clotting ability over the areas of the body not undergoing thrombosis. Selective pulmonary anti-coagulation can also be utilized to treat other pulmonary diseases marked by pulmonary thrombosis such as pulmonary hypertension, lung transplant rejection and others.

In a disease like chronic obstructive pulmonary disease, which is often marked by shortness of breath, the plant-derived component or components of the present invention can be co-administered to boost the effective concentration and potency of drugs to relax airway smooth muscles such as long lasting beta-2 agonists such as salmeterol or formoterol.

Many pulmonary diseases are often marked by a decrease in glutathione (GSH), a powerful antioxidant. The plant-derived component or components of the present invention can be co-administered with N-Acetylcysteine (NAC), a glutathione precursor, in diseases like pulmonary fibrosis, PAH, ALI, and other pulmonary disorders to boost GSH production and scavenge reactive oxidants often found in pulmonary diseases. GSH may also serve to dampen the inflammatory immune response by binding to triggering receptor expressed on myeloid cells 1 (TREM1) and diminishing monocyte/macrophage- and neutrophil-mediated inflammatory responses. Co-administration of CAR with NAC can serve to lessen the severe inflammatory immune response that often characterizes severe pulmonary and fibrotic diseases like ALI, pulmonary hypertension, autoimmune diseases and many other conditions.

Treatments for pulmonary diseases like pulmonary fibrosis, PAH and ALI can also be improved by co-administering the plant-derived component or components of the present invention with TGF-beta inhibitors like decorin.

In pulmonary hypertension, pulmonary fibrosis and other pulmonary diseases, the benefits of endothelin (ET-1) receptor antagonists, prostacyclin derivatives, phosphodiesterase type 5 inhibitors can be increased for patients through the co-administration with the plant-derived component or components of the present invention.

Other pulmonary and fibrotic disease treatments such as Ketoconazole which inhibits thromboxane and leukotriene synthesis can be improved in its efficacy while minimizing side effects through co-administration with the plant-derived component or components of the present invention.

The term “treating” refers to inhibiting, preventing or arresting the development of a pathology (disease, disorder or condition) and/or causing the reduction, remission, or regression of a pathology. Those of skill in the art will understand that various methodologies and assays can be used to assess the development of a pathology, and similarly, various methodologies and assays may be used to assess the reduction, remission or regression of a pathology.

As used herein, the term “preventing” refers to keeping a disease, disorder or condition from occurring in a subject who may be at risk for the disease, but has not yet been diagnosed as having the disease.

As used herein, the term “subject” includes mammals, preferably human beings, male or female, at any age or gender, who suffer from the pathology. Preferably, this term encompasses individuals who are at risk to develop the pathology (e.g., above 65 of age, exposed to airborne pollutants, particulates, toxic fumes, allergens).

The composition of matter comprising the component(s) (a plant species or genus thereof-derived component selected from the group consisting of a plant part, extract thereof, fraction thereof, active ingredient thereof, synthetic analog thereof, mimetic thereof or combination thereof, wherein said component is capable of ameliorating symptoms of NMRD) of the present invention can be administered to the subject per se, or in a pharmaceutical composition where it is mixed with suitable carriers or excipients.

As used herein a “pharmaceutical composition” refers to a preparation of one or more of the active ingredients described herein with other chemical components such as physiologically suitable carriers and excipients. The purpose of a pharmaceutical composition is to facilitate administration of a compound to an organism.

Herein the term “active ingredient” refers to the composition of matter comprising the components accountable for the biological effect.

Hereinafter, the phrases “physiologically acceptable carrier” and “pharmaceutically acceptable carrier” which may be interchangeably used refer to a carrier or a diluent that does not cause significant irritation to an organism and does not abrogate the biological activity and properties of the administered compound. An adjuvant is included under these phrases.

Herein the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils and polyethylene glycols.

Techniques for formulation and administration of drugs may be found in “Remington's Pharmaceutical Sciences,” Mack Publishing Co., Easton, PA, latest edition, which is incorporated herein by reference.

Suitable routes of administration may, for example, include oral, rectal, transmucosal, especially transnasal, intestinal or parenteral delivery, including intramuscular, subcutaneous and intramedullary injections as well as intrathecal, direct intraventricular, intracardiac, e.g., into the right or left ventricular cavity, into the common coronary artery, intravenous, intraperitoneal, intranasal, or intrapulmonary or intraocular injections.

In various exemplary embodiments of the invention, the composition is provided as a pharmaceutical or dietary supplement dosage form suitable for oral administration. Dosage forms suitable for oral administration include tablets, soft capsules, hard capsules, pills, granules, powders, emulsions, suspensions, sprays, syrups and pellets. In various other embodiments of the invention, the composition is provided as a pharmaceutical dosage form suitable for parenteral administration such as liquid formulations for administration as drops or by injection, or as solid or semisolid dosage forms for suppositories.

Conventional approaches for drug delivery to the central nervous system (CNS) include: neurosurgical strategies (e.g., intracerebral injection or intracerebroventricular infusion); molecular manipulation of the agent (e.g., production of a chimeric fusion protein that comprises a transport polypeptide that has an affinity for an endothelial cell surface molecule in combination with an agent that is itself incapable of crossing the BBB) in an attempt to exploit one of the endogenous transport pathways of the BBB; pharmacological strategies designed to increase the lipid solubility of an agent (e.g., conjugation of water-soluble agents to lipid or cholesterol carriers); and the transitory disruption of the integrity of the BBB by hyperosmotic disruption (resulting from the infusion of a mannitol solution into the carotid artery or the use of a biologically active agent such as an angiotensin polypeptide). However, each of these strategies has limitations, such as the inherent risks associated with an invasive surgical procedure, a size limitation imposed by a limitation inherent in the endogenous transport systems, potentially undesirable biological side effects associated with the systemic administration of a chimeric molecule comprised of a carrier motif that could be active outside of the CNS, and the possible risk of brain damage within regions of the brain where the BBB is disrupted, which renders it a suboptimal delivery method.

Alternately, one may administer the pharmaceutical composition in a local rather than systemic manner, for example, via injection of the pharmaceutical composition directly into a tissue region of a patient.

Pharmaceutical compositions of some embodiments of the invention may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with some embodiments of the invention thus may be formulated in conventional manner using one or more physiologically acceptable carriers comprising excipients and auxiliaries, which facilitate processing of the active ingredients into preparations which, can be used pharmaceutically. Proper formulation is dependent upon the route of administration chosen.

For injection, the active ingredients of the pharmaceutical composition may be formulated in aqueous solutions, preferably in physiologically compatible buffers such as Hank's solution, Ringer's solution, or physiological salt buffer. For transmucosal administration, penetrants appropriate to the barrier to be permeated are used in the formulation. Such penetrants are generally known in the art.

For oral administration, the pharmaceutical composition can be formulated readily by combining the active compounds with pharmaceutically acceptable carriers well known in the art. Such carriers enable the pharmaceutical composition to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carbomethylcellulose; and/or physiologically acceptable polymers such as polyvinylpyrrolidone (PVP). If desired, disintegrating agents may be added, such as cross-linked polyvinyl pyrrolidone, agar, or alginic acid or a salt thereof such as sodium alginate.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active compound doses.

Pharmaceutical compositions which can be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active ingredients may be dissolved or suspended in suitable liquids, such as fatty oils, liquid paraffin, or liquid polyethylene glycols. In addition, stabilizers may be added. All formulations for oral administration should be in dosages suitable for the chosen route of administration.

For buccal administration, the compositions may take the form of tablets or lozenges formulated in conventional manner.

In specific embodiments, the components and/or compositions of the invention are provided in form suitable for administration by inhalation or nasal administration.

For administration by nasal inhalation, the active ingredients for use according to some embodiments of the invention are conveniently delivered in the form of an aerosol spray presentation from a pressurized pack or a nebulizer with the use of a suitable propellant, e.g., dichlorodifluoromethane, trichlorofluoromethane, dichloro-tetrafluoroethane or carbon dioxide. In the case of a pressurized aerosol, the dosage unit may be determined by providing a valve to deliver a metered amount. Capsules and cartridges of, e.g., gelatin for use in a dispenser may be formulated containing a powder mix of the compound and a suitable powder base such as lactose or starch.

The pharmaceutical composition described herein may be formulated for parenteral administration, e.g., by bolus injection or continuous infusion. Formulations for injection may be presented in unit dosage form, e.g., in ampoules or in multidose containers with optionally, an added preservative. The compositions may be suspensions, solutions or emulsions in oily or aqueous vehicles, and may contain formulatory agents such as suspending, stabilizing and/or dispersing agents.

Pharmaceutical compositions for parenteral administration include aqueous solutions of the active preparation in water-soluble form. Additionally, suspensions of the active ingredients may be prepared as appropriate oily or water based injection suspensions. Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl cellulose, sorbitol or dextran. Optionally, the suspension may also contain suitable stabilizers or agents which increase the solubility of the active ingredients to allow for the preparation of highly concentrated solutions.

Alternatively, the active ingredient may be in powder form for constitution with a suitable vehicle, e.g., sterile, pyrogen-free water based solution, before use.

The pharmaceutical composition of some embodiments of the invention may also be formulated in rectal compositions such as suppositories or retention enemas, using, e.g., conventional suppository bases such as cocoa butter or other glycerides.

Pharmaceutical compositions suitable for use in context of some embodiments of the invention include compositions wherein the active ingredients are contained in an amount effective to achieve the intended purpose. More specifically, a therapeutically effective amount means an amount of active ingredients (composition of matter comprising the components accountable for the biological effect) effective to prevent, alleviate or ameliorate symptoms of a disorder (e.g., NMRD) or prolong the survival of the subject being treated.

Determination of a therapeutically effective amount is well within the capability of those skilled in the art, especially in light of the detailed disclosure provided herein.

For example, any in vivo or in vitro method of evaluating the severity of NMRD symptoms may be employed.

For any preparation used in the methods of the invention, the therapeutically effective amount or dose can be estimated initially from in vitro and cell culture assays. For example, a dose can be formulated in animal models to achieve a desired concentration or titer. Such information can be used to more accurately determine useful doses in humans.

Toxicity and therapeutic efficacy of the active ingredients described herein can be determined by standard pharmaceutical procedures in vitro, in cell cultures or experimental animals. The data obtained from these in vitro and cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition. (See e.g., Fingl, et al., 1975, in “The Pharmacological Basis of Therapeutics”, Ch. 1 p. 1).

Dosage amount and interval may be adjusted individually to provide the active ingredient at a sufficient amount to induce or suppress the biological effect (minimal effective concentration, MEC). The MEC will vary for each preparation, but can be estimated from in vitro data. Dosages necessary to achieve the MEC will depend on individual characteristics and route of administration. Detection assays can be used to determine plasma concentrations.

Depending on the severity and responsiveness of the condition to be treated, dosing can be of a single or a plurality of administrations, with course of treatment lasting from several days to several weeks or until cure is effected or diminution of the disease state is achieved.

The amount of a composition to be administered will, of course, be dependent on the subject being treated, the severity of the affliction, the manner of administration, the judgment of the prescribing physician, etc.

Compositions of some embodiments of the invention may, if desired, be presented in a pack or dispenser device, such as an FDA approved kit, which may contain one or more unit dosage forms containing the active ingredient. The pack may, for example, comprise metal or plastic foil, such as a blister pack. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert. Compositions comprising a preparation of the invention formulated in a compatible pharmaceutical carrier may also be prepared, placed in an appropriate container, and labeled for treatment of an indicated condition, as is further detailed above.

In another embodiment, the invention provides a nutritional or dietary compositions in the form of foods or beverages, which comprise the component(s) described herein. These foods or beverages comprise various exemplary embodiments of the inventive compositions. These foods or beverages can be prepared or provided as cereals, baby foods, healthy foods, or food for specified health uses such as solid food like chocolate or nutritional bars, semisolid food like cream or jam, or gel; and also as beverages. Specific and non-limiting examples of such food or beverage items include refreshing beverages, lactic acid bacteria beverages, drops, candies, chewing gum, chocolate, gummy candy, yoghurts, ice creams, puddings, soft adzuki bean jellies, jellies, cookies and the like.

The present teachings further envisage treating with other anti-viral drugs or anti-inflammatory drugs or anti-coagulants as separate treatments or in a co-formulation.

Without being limited to NMRD but for the sake of example, according to a specific embodiment, the antiviral drug is selected from the group consisting of remdesivir, an interferon, ribavirin, adefovir, tenofovir, acyclovir, brivudin, cidofovir, fomivirsen, foscarnet, ganciclovir, penciclovir, amantadine, rimantadine and zanamivir.

Also contemplated are plasma treatments from infected persons who survived and/or anti-HIV drugs such as lopinavir and ritonavir, as well as chloroquine.

Specific examples of drugs that are routinely used for the treatment of NMRD include, but are not limited to, anti-inflammatories, bronchodilators such as beta-2-agonists and anticholinergics, corticosteroids, phosphodieaterase-4-inhibitors, theophylline, antibiotics and antivirals could be the drug treatment options for NMRD.

As used herein the term “about” refers to ±10%

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.
As used herein the term “viral entry mechanism” refers to viral proteins that mediate entry into cells. The viral entry mechanism proteins include attachment proteins and other proteins that are required for entry of non-enveloped and enveloped viruses into cells. Different viruses use different entry proteins, however, both non-enveloped and enveloped viruses share the same two main steps and routes of virus entry; (1) attachment to cell-surface receptors (2) conformational changes of the viral entry proteins or the host-cell receptors, the viral entry can occur either by penetration of the cell membrane (for non-enveloped viruses) or fusion (for enveloped viruses) to the cell membrane (see “Virus entry: molecular mechanisms and biomedical applications”, Dimitrov, 2004)

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non-limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Maryland (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, CT (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, CA (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### Example 1

### Assays for Amelioration of NMRD

Many cell-based, in-vitro systems for evaluation of conditions comprising NMRD are available, in addition to traditional in-vivo animal models. To model NMRD, primary as well as cell lines of airway-associated cells such as pleural mesothelial cells (PMC), small airway epithelial cells (SAEC), bronchial epithelial cells (BEC), nasal epithelial cells, and bronchial fibroblasts are cultured and exposed to chemical/pathogenic insults. Cell responses, and in particular inflammatory processes, senescence and cell activation are determined in the presence or absence of added compositions and/or components of the invention, in order to evaluate the ability of the compositions and/or components of the invention to reduce or otherwise modify the effect of the insults on the cells. Cells can be propagated in 2-D or 3-D cultures.

For example, cells for in-vitro modeling of asthma include human pulmonary smooth muscle cells and airway epithelial cells. Cells for modeling of pulmonary fibrosis include pleural mesothelial cells. COPD can be modeled in vitro with bronchial fibroblasts, and asbestos/silica-induced damage can be evaluated using pleural mesothelial cells.

In an exemplary cell-based assay of fibrotic respiratory disease (e.g. COPD, asthma, IPF), fibroblast-to-myofibroblast transformation and epithelial-to-mesenchymal transition are assayed in normal lung fibroblasts and small airway epithelial cells.

Primary human lung fibroblasts or SAEC cells are induced into transformation with the pro-fibrotic growth factor TGFbeta-1, and markers of the different phenotypes (aSMA, collagen-I, fibronectin, E-cadherin) are evaluated. Anti-fibrotic effects of the compositions and/or components of the invention on the extent and character of transformation are evaluated.

Animal Models of Non-Malignant Respiratory Disease

Animal models for NMRD include induced animal models and naturally occurring animal models of the respiratory diseases and conditions.

Induced animal models suitable for assessing efficacy of the compositions and components of the invention include, but are not limited to:
COPD: Rodents, exposure to cigarette smoke, assessment of vascular wall and airway remodeling;
EMPHYSEMA: Rodents, exposure to elastin-degrading elastase, transgenic overexpression of collagenase;
LOWER RESPIRATORY INFECTION: Rodents, cohabitation, aerosolization, nebulizer, direct airway installation;
PULMONARY FIBROSIS: Rodents, environmental exposure (silicosis, asbestosis, allergens, Bleomycin induction, hypochlorous acid HOCl, hapteninduced lung fibrosis;
ASTHMA: Rodents, repeated challenge with allergens (leading to goblet-cell hyperplasia, airway inflammation and hyperresponsiveness (remodeling), exposure to dust mites.

A few naturally occurring animal models for NMRD are known and may be suitable for assessment of the effects of the compositions and/or components of the invention: IPF susceptibility is documented in certain breeds cats, dogs, horses and donkeys, and the asthma-like Hyperreactive Airway Disease is well known in horses and cats.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

### Example 2

### Assays for Amelioration of SARS-CoV-2 by Plant Components

COVID-19 is caused by severe acute respiratory syndrome coronavirus 2 (SARS-CoV-2). The cell membrane ACE-2 receptor is an attachment and entry site for SARS-CoV-2. The ACE-2 receptor is a type I transmembrane metallocarboxypeptidase with homology to ACE, an enzyme long-known to be a key player in the Renin-Angiotensin system (RAS) and a target for the treatment of hypertension. There is evidence that SARS-CoV-2 utilizes ACE-2 as a cellular entry receptor. Zhou et al. showed that SARS-CoV-2 could use ACE-2 from humans, Chinese horseshoe bats, civet cats, and pigs to gain entry into ACE-2-expressing HeLa cells (See Zhou, P., Yang, X L., Wang, X G. et al. A pneumonia outbreak associated with a new coronavirus of probable bat origin. Nature 579, 270-273 (2020)). The spike (S) protein of SARS-CoV-2, which plays a key role in the receptor recognition and cell membrane fusion process, is composed of two subunits, S1 (120 kDa) and S2 (80 kDa). The S1 subunit contains a receptor-binding domain that recognizes and binds to the host receptor angiotensin-converting enzyme 2 (ACE-2). The S2 subunit mediates viral cell membrane fusion by forming a six-helical bundle via the two-heptad repeat domain (see Huang, Y., Yang, C., Xu, Xf. et al. Structural and functional properties of SARS-CoV-2 spike protein: potential antivirus drug development for COVID-19. Acta Pharmacol Sin 41, 1141-1149 (2020)). Interfering, attenuating, impairing the function of the S1 and S2 subunits will eventually lead to an attenuated, impaired and less infective virus.

### Viral Protein Digestion Assays

The materials used in all the following viral protein digestion assays are disclosed in table 1.

TABLE 1

| | Company | cat # |
|---|---|---|
| Recombinant SARS-Cov-2 S1 Subunit | KavBiotech | 230-30161 |
| Recombinant SARS-Cov-2 S2 Subunit | BaySiotack | 230-30163 |
| Recombinant SARS-Cov-2 Nucleocapsid | RayBiotech | 230-30154 |
| Proteinase K | Sigma | P4850 |
| Instanst Blue | Expedeon | ISB1L |
| DMSO | SIGMA | D2650 |
| Criterion ™ TGX Stain-Free Precast Midi | BIDRAD | 5671084 |
| Gels 4-15%, 18 wells, 30 μl | | |
| 10X Tris/Glycine/SDS | BIORAD | 161-0772 |
| 4X Laemmli Sample Buffer | BIORAD | 1610747 |
| B-Mercaptoethanol 14.2M | SIGMA | M3148 |
| Precision protein standard | BIORAD | 161-0373 |

Each of the tested plant based treatment was numbered as disclosed in table 2 and the tested combinations complexes were classified as disclosed in table 3

TABLE 2

| Treatment | |
|---|---|
| No. | Name |
| 1 | Oregano Oil |
| 2 | Thyme Oil |
| 3 | Nigela Sativa |
| 4 | Sumac Oil |
| 5 | Sesame Oil |
| 6 | Olibanum Oil |

TABLE 3

| | |
|---|---|
| Complex A | Oils 1 + 2 + 3 |
| Complex B | Oils 1 + 2 + 3 + 4 |
| Complex C | Oils 1 + 2 + 3 + 4 + 5 |
| Complex D | Oils 1 + 2 + 3 + 4 + 5 + 6 |

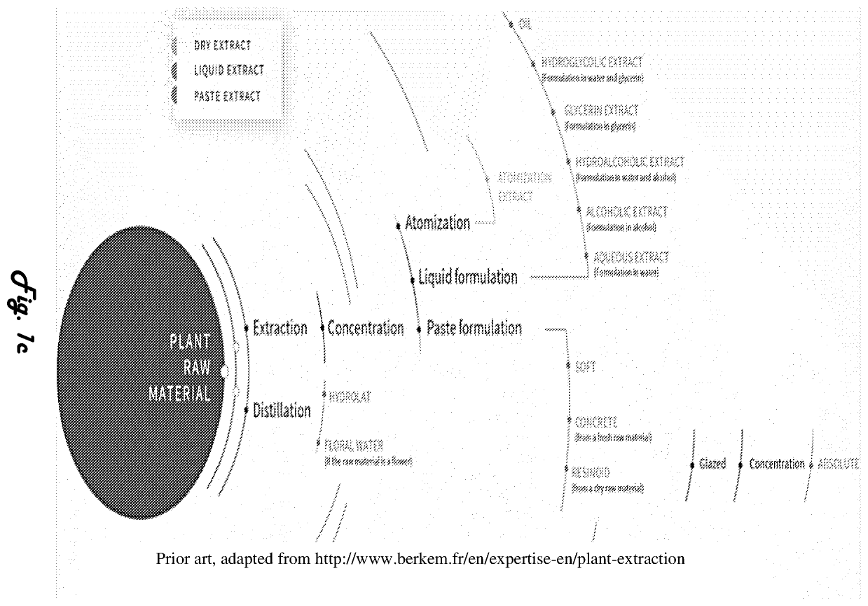

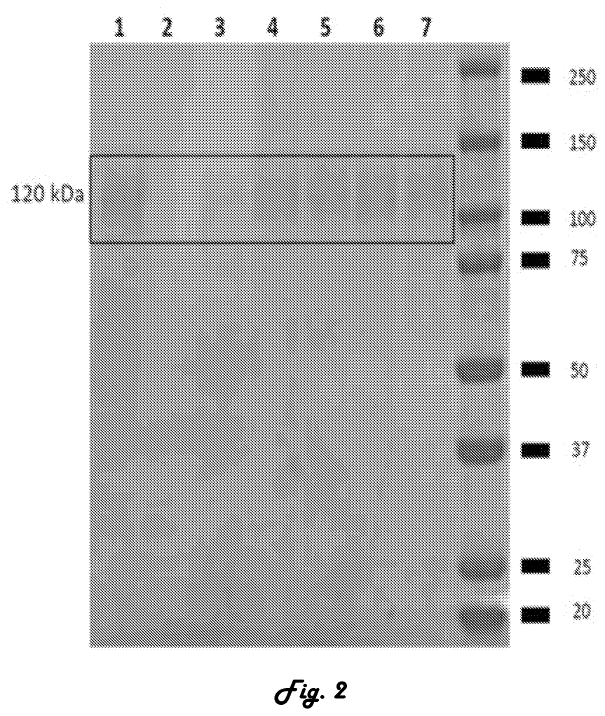

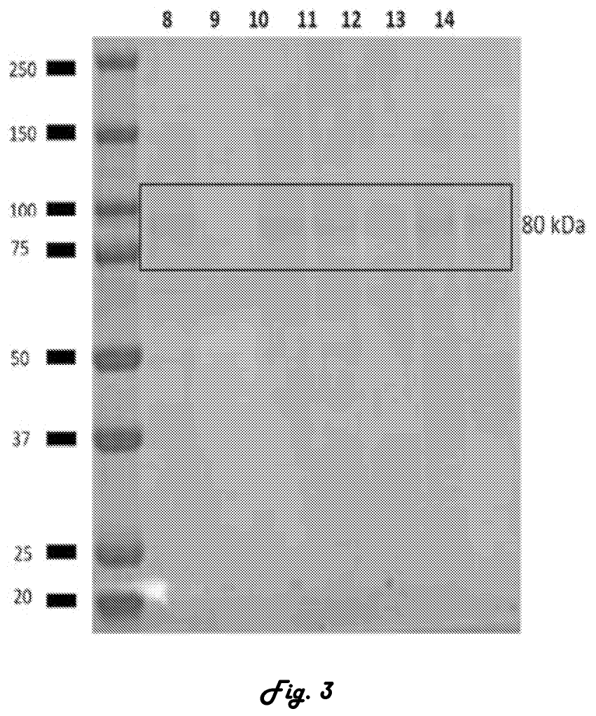

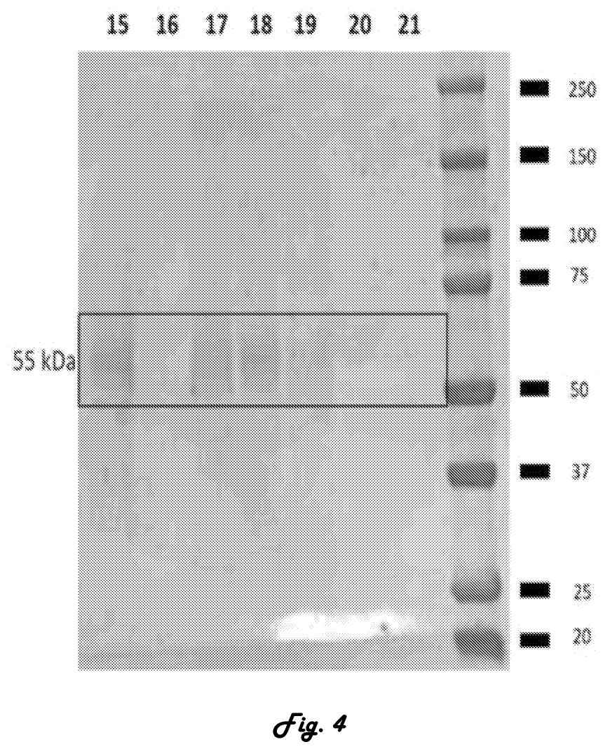

Oils mixtures were prepared by mixing equal amounts of each oil. The mix was then diluted 1:2 with DMSO, to acquire a solution of 50% DMSO, 50% Oil mix and the final reaction concentration was 5% oil mix, 5% DMSO.
For each assay reaction, 1 μg protein per reaction was incubated with 3 μl of the oil mixture at final reaction volume of 30 μl. The reaction was incubated for 6 hours at 37° C.
Following incubation, the reaction was stopped by adding 10 μl/reaction of sample buffer 4× and incubation 10 minutes at 72° C. Samples were then run in 4-15% TGX Criterion Gel (BIORAD) for 50 minutes at 200 Volt. Following run, the gel was incubated for 1 hour with Instant Blue reagent (Expedeon) and further washed with water until distinct bands were observed.
Densitometry was preformed, pictures were analyzed with ImageJ software.
The protein digestion assay was conducted as disclosed in table 4. SARS-CoV-2 S1 subunit, SARS-CoV-2 S2 subunit, SARS-CoV-2 Nucleocapsid protein and a negative control with no protein, were incubated with different plant oils or combinations for 6 h at 37° c. and subsequently run on SDS-page, stained with “instant blue” for the presence of proteins in the gel. The untreated control appeared at the expected molecular weight and the effect of different treatments were compared to this control (see FIGS. 2-4). Significant disappearance pf the protein was observed following treatment with the protease proteinase K.

TABLE 4

| No # | Protein | Treatment | Incubation | μg/lane | Expected MW (KDa) |
|---|---|---|---|---|---|
| 1 | SARS-CoV-2.51 Subunit | Non treated—control | W/O Incubation | 1 μg | 120 kDa |
| 2 | | Positive control—Proteinase K | 6 hours—37° C. | | |
| 3 | | Complex B | | | |
| 4 | | Complex C | | | |
| 5 | | Complex D | | | |
| 6 | | Negative control—DMSO | | | |
| 7 | | Non treated—control | | | |
| 8 | SARS-CoV-2.52 Subunit | Non treated—control | W/O Incubation | 1 μg | 80 kDa |
| 9 | | Positive control—Proteinase K | 6 hours—37° C. | | |
| 10 | | Complex B | | | |
| 11 | | Complex C | | | |
| 12 | | Complex D | | | |
| 13 | | Negative control—DMSO | | | |
| 14 | | Non treated—control | | | |
| 15 | SARS-CoV-2 Nucleocapsid | Non treated—control | W/O Incubation | 1 μg | 55 kDa |
| 16 | | Positive control—Proteinase K | 6 hours—37° C. | | |
| 17 | | Complex B | | | |
| 18 | | Complex C | | | |
| 19 | | Complex D | | | |
| 20 | | Negative control—DMSO | | | |
| 21 | | Non treated—control | | | |
| 22 | No protein | Complex B | 6 hours—37° C. | | |
| 23 | | Complex C | | | |
| 24 | | Complex D | | | |

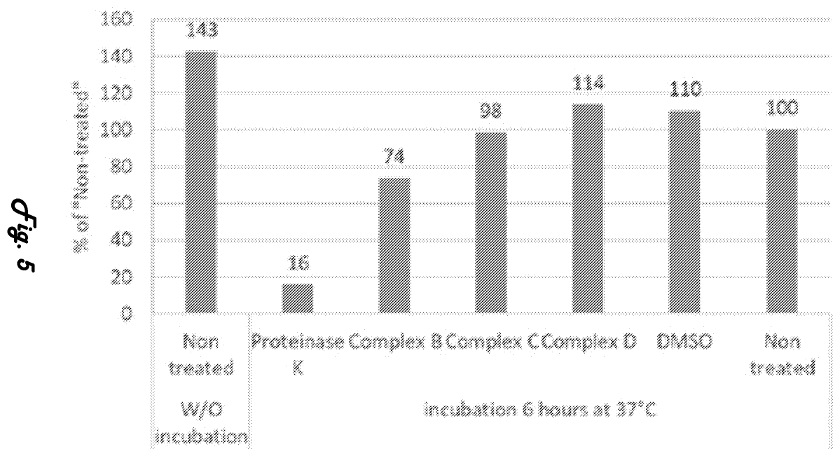

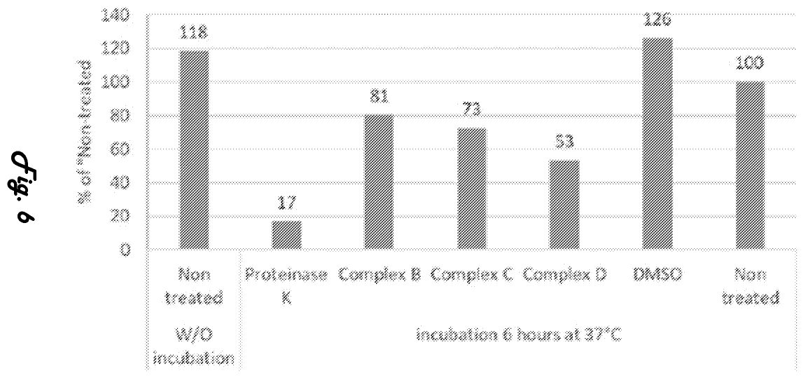

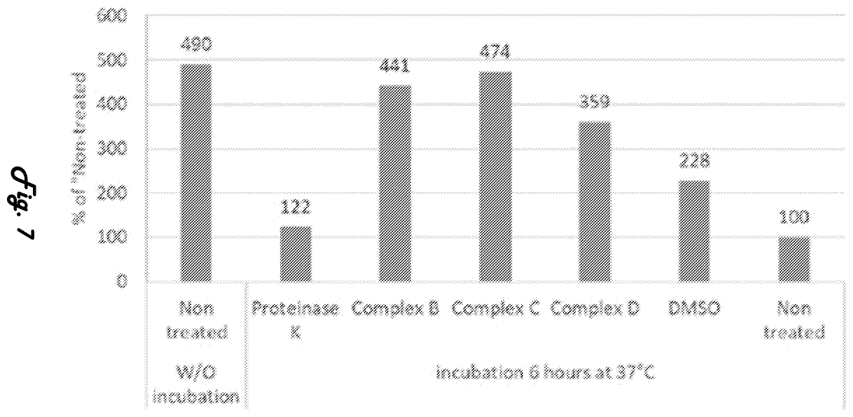

Densitometry of the SARS-CoV-2 S1 subunit, SARS-CoV-2 S2 subunit, SARS-CoV-2 and the Nucleocapsid protein assays disclose that although the Nucleocapsid protein underwent little to no digestion with either of the tested treatments as compared to the protein K treatment the two SARS-CoV-2 subunits S1 and S2 underwent a substantial digestion (see FIGS. 5-7 respectively).
To conclude, the viral protein digestion assay demonstrates that there is a significant digestion of the S1 and S2 subunits without destroying the Nucleocapsid protein.

### Recombinant SARS-Cov-2 S1 Subunit

Following incubation of the protein with a mix prepared from equal volumes from items 1+2+3+4 (complex B), a 26% reduction in the protein signal was observed.

### Recombinant SARS-Cov-2 S2 Subunit Protein

Following incubation of the protein with a mix prepared from equal volumes from items 1+2+3+4 (complex B), a 19% reduction in the protein signal was observed.
Following incubation of the protein with a mix prepared from equal volumes from items 1+2+3+4+5 (complex C), a 27% reduction in the protein signal was observed
Following incubation of the protein with a mix prepared from equal volumes from items 1+2+3+4+5+6 (complex D), a 47% reduction in the protein signal was observed.
These significant digestion rates of both S1 and S2 subunits of the spike protein are likely to result in the subsequent attenuation of the Coronavirus cell attachment and internalization mechanism. It is clear that attenuating the virus cell attachment and internalization mechanism, disease levels and viral load can be reduced in an infected subject, or prevent an uninfected subject from getting infected.
It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

1. A method of reducing the infectivity of a non-malignant respiratory disease (NMRD) virus by modifying the viral entry mechanism proteins in a subject, the method comprising administering to the subject an effective amount of a composition of claim 5.

2. A method of reducing the infectivity of a virus by modifying the viral entry mechanism proteins in a subject, the method comprising administering to the subject an effective amount of a composition of claim 5.

3. A method of treating non-malignant respiratory disease (NMRD) in a subject in need thereof, the method comprising administering to the subject an effective amount of a plant species or genus thereof-derived component selected from a plant part, extract thereof, fraction thereof, active ingredient thereof, synthetic analog thereof, mimetic thereof or combination thereof, wherein said component is capable of ameliorating symptoms of NMRD and wherein said plant species is selected from Nigella sativa, Thymus capitatus, Thymus vulgaris, Origanum syriacum, Thymbra spicata, Satujera thymbra, Sesamum indicum, Rhus coriaria, Gynostemma pentaphyllum, Boswellia sacra and Panax ginseng.

4. A vaccine against NMRD comprising an effective amount of a plant species or genus thereof-derived component selected from a plant part, extract thereof, fraction thereof, active ingredient thereof, synthetic analog thereof, mimetic thereof or combination thereof, wherein said component is capable of ameliorating symptoms of NMRD and wherein said plant species is selected from Nigella sativa, Thymus capitatus, Thymus vulgaris, Origanum syriacum, Thymbra spicata, Satujera thymbra, Sesamum indicum, Rhus coriaria, Gynostemma pentaphyllum, Boswellia sacra and Panax ginseng.

5. A pharmaceutical composition comprising an effective amount of a plant species or genus thereof-derived component selected from a plant part, extract thereof, fraction thereof, active ingredient thereof, synthetic analog thereof, mimetic thereof or combination thereof, wherein said component is capable of ameliorating symptoms of NMRD and wherein said plant species is selected from Nigella sativa, Thymus capitatus, Thymus vulgaris, Origanum syriacum, Thymbra spicata, Satujera thymbra, Sesamum indicum, Rhus coriaria, Gynostemma pentaphyllum, Boswellia sacra and Panax ginseng for use in preventing or treating NMRD.

6. The composition of claim 5 comprising at least 2 of said plant species or genus thereof-derived components.

7. A food supplement comprising a combination of at least 2 of a plant species or genus thereof-derived component selected from a plant part, extract thereof, fraction thereof, active ingredient thereof, synthetic analog thereof, mimetic thereof or combination thereof, wherein said component is capable of ameliorating symptoms of NMRD and wherein said plant species is selected from Nigella sativa, Thymus capitatus, Thymus vulgaris, Origanum syriacum, Thymbra spicata, Satujera thymbra, Sesamum indicum Rhus coriaria, Gynostemma pentaphyllum, Boswellia sacra and Panax ginseng.

8. The method of claim 1, wherein said NMRD is selected from pulmonary hypertension, interstitial lung disease, acute lung injury (ALI), acute respiratory distress syndrome (ARDS), sarcoidosis of the lung, bronchiectasis, asbestosis, berylliosis, silicosis, anthracosis, Histiocytosis X, pneumotitis, smoker's lung, bronchiolitis obliterans, lung scarring due to tuberculosis and pulmonary fibrosis, chronic obstructive pulmonary disease, pneumoconiosis, traumatic pulmonary injury, pulmonary infections and pulmonary manifestations of connective tissue diseases, including systemic lupus erythematosus, rheumatoid arthritis, scleroderma, polymyositis and dermatomyositis.

9. The method of claim 1, wherein said NMRD is Chronic Obstructive Pulmonary Disease (COPD), Idiopathic Pulmonary Fibrosis (IPF) or Asthma.

10. The method of claim 1, wherein said symptoms are selected from coughing, shortness of breath, wheezing, difficulty in breathing, inflammation of the airways, chest tightness and exercise intolerance.

11. The method of claim 1, wherein said component comprises at least 2 components.

12. The method of claim 1, wherein said component comprises at least 3 components.

13. The method of claim 1, wherein said component comprises at least 4 components.

14. The method of claim 1, wherein said component comprises at least 5 components.

15. The method of claim 1, wherein said component comprises 5-10 components.

16. The method of claim 1, wherein said component comprises thymoquinone or an analog thereof.

17. The method of claim 1, wherein said component comprises thymol or an analog thereof.

18. The method of claim 1, wherein said component comprises carvacrol or an analog thereof.

19. The method of claim 1, wherein said component comprises bromelain or an analog thereof.

20. The method of claim 1, wherein said component comprises pineapple extract comprising bromelain or an analog thereof.

21. The method of claim 1, wherein said component comprises tryptophan or an analog thereof, or extract of a plant containing tryptophan.

22. The method of claim 1 wherein said plant species or genus thereof-derived component further comprises “Beduin Tea” comprising Rose Leaves Micromeria fruticose, Salvia, cymbopgon (Citral) Aloysia, Verbena officinalis, Origanum majorana, and menthe.

23. The method of claim 1 wherein said plant species or genus thereof-derived component further comprises “Beduin Tea” comprising Thyme, sage, cardamom, cinnamon, black tea, habuk, Marmaya.
